(19) United States
(12) Patent Application Publication
(10) Pub. No.: US 2026/0186877 A1
(43) Pub. Date: Jul. 2, 2026

(54) FAILURE PREDICTION VIA GRAPH REPRESENTATION OF ERROR CODES

(71) Applicant: GE Precision Healthcare LLC, Waukesha, WI (US)

(72) Inventors: Karim Choukri, Paris (FR); Harsha Aeron, Bangalore (IN); Sourav Banerjee, Burdwan (IN)

(21) Appl. No.: 19/419,442

(22) Filed: Dec. 15, 2025

Related U.S. Application Data
(60) Provisional application No. 63/740,175, filed on Dec. 30, 2024.

(51) Int. Cl. G06F 11/00; G16H 40/40; G16H 40/67

(52) U.S. Cl. CPC G06F 11/004; G16H 40/40; G16H 40/67; G06F 2201/805

(57) ABSTRACT

Systems/techniques that facilitate failure prediction via graph representation of error codes are provided. A system can access a medical scanner having a plurality of instrumented hardware parts, each monitored by a respective set of sensors and configured to produce respective error codes in response to respective combinations of measurements from its sensors. The system can construct a sequence of order-of-receipt knowledge graphs based on monitoring the instrumented hardware parts over a sequence of time intervals, each node of each graph representing a respective error code producible by the instrumented hardware parts, and each edge of each graph representing an order in which two respective error codes are produced by the instrumented hardware parts during a respective time interval. The system can generate a maintenance prediction for at least one instrumented hardware part, based on executing a deep learning neural network on the sequence of order-of-receipt knowledge graphs.

### CROSS-REFERENCE TO RELATED APPLICATION

This application claims priority to U.S. Provisional Patent Application No. 63/740,175 filed on Dec. 30, 2024, entitled “SYSTEMS AND METHODS FOR FAILURE PREDICTION IN MEDICAL DEVICES”. The entirety of the aforementioned provisional application is incorporated by reference herein.

### TECHNICAL FIELD

The subject disclosure relates generally to failure prediction of medical imaging scanners.

### BACKGROUND

Medical imaging scanners can suffer operational failures. The occurrence of such failures can negatively affect medical patients. To avoid such negative effects, such failures can be predicted in advance via artificial intelligence.

Systems or techniques for increasing the accuracy or reliability of such failure prediction can be desired.

### SUMMARY

The following presents a summary to provide a basic understanding of one or more embodiments. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus or computer program products that facilitate failure prediction via graph representation of error codes are described.

According to one or more embodiments, a system is provided. The system can access a medical imaging scanner, wherein: the medical imaging scanner can include a plurality of instrumented hardware parts; each of the plurality of instrumented hardware parts can be monitored by a respective set of physical sensors; and each of the plurality of instrumented hardware parts can be configured to produce one or more respective electronic error codes in response to one or more respective combinations of measurements from its physical sensors. The system can construct a sequence of order-of-receipt knowledge graphs based on monitoring the plurality of instrumented hardware parts over a sequence of time intervals, wherein: a first order-of-receipt knowledge graph of the sequence of order-of-receipt knowledge graphs can correspond to a first time interval of the sequence of time intervals; each node of the first order-of-receipt knowledge graph can represent a respective electronic error code producible by the plurality of instrumented hardware parts; and each edge of the first order-of-receipt knowledge graph can represent an order in which two respective electronic error codes are produced by the plurality of instrumented hardware parts during the first time interval. The system can estimate a remaining useful life of or recommend a maintenance task for at least one of the plurality of instrumented hardware parts, based on executing a deep learning neural network on the sequence of order-of-receipt knowledge graphs.

According to one or more embodiments, a computer-implemented method is provided. The computer-implemented method can include accessing, by a device operatively coupled to a processor, a medical imaging scanner, wherein: the medical imaging scanner can have a plurality of instrumented hardware parts; each of the plurality of instrumented hardware parts can be monitored by a respective set of physical sensors; and each of the plurality of instrumented hardware parts can be configured to produce one or more respective electronic error codes in response to one or more respective combinations of measurements from its physical sensors. The computer-implemented method can include constructing, by the device, a sequence of order-of-receipt knowledge graphs based on monitoring the plurality of instrumented hardware parts over a sequence of time intervals, wherein: a first order-of-receipt knowledge graph of the sequence of order-of-receipt knowledge graphs can correspond to a first time interval of the sequence of time intervals; each node of the first order-of-receipt knowledge graph can represent a respective electronic error code producible by the plurality of instrumented hardware parts; and each edge of the first order-of-receipt knowledge graph can represent an order in which two respective electronic error codes are produced by the plurality of instrumented hardware parts during the first time interval. The computer-implemented method can include estimating or recommending, by the device, a remaining useful life of or a maintenance task for at least one of the plurality of instrumented hardware parts, based on executing a deep learning neural network on the sequence of order-of-receipt knowledge graphs.

According to one or more embodiments, a computer program product facilitating failure prediction via graph representation of error codes is provided. The computer program product can include a non-transitory computer-readable memory having program instructions embodied therewith. The program instructions can be executable by a processor to cause the processor to access a machine, wherein: the machine can have a plurality of instrumented hardware parts; each of the plurality of instrumented hardware parts can be monitored by a respective set of physical sensors; and each of the plurality of instrumented hardware parts can be configured to produce one or more respective electronic error codes in response to one or more respective combinations of measurements from its physical sensors. The program instructions can be executable by the processor to cause the processor to construct a sequence of knowledge graphs based on monitoring the plurality of instrumented hardware parts over a sequence of time intervals, wherein: a first knowledge graph of the sequence of knowledge graphs can correspond to a first time interval of the sequence of time intervals; each node of the first knowledge graph can represent a respective electronic error code producible by the plurality of instrumented hardware parts; and each edge of the first knowledge graph can represent an order in which two respective electronic error codes are produced by the plurality of instrumented hardware parts during the first time interval. The program instructions can be executable by the processor to cause the processor to estimate or recommend a remaining useful life of or a maintenance task for at least one of the plurality of instrumented hardware parts, based on executing a deep learning neural network on the sequence of knowledge graphs.

### DESCRIPTION OF THE DRAWINGS

FIG. 1 illustrates a block diagram of an example, non-limiting system that facilitates failure prediction via graph representation of error codes in accordance with one or more embodiments described herein.

FIG. 2 illustrates an example, non-limiting block diagram of instrumented hardware parts of a medical imaging scanner in accordance with one or more embodiments described herein.

FIG. 3 illustrates a block diagram of an example, non-limiting system including an order-of-receipt knowledge graph sequence, a retrospective time span, and a sliding time window that facilitates failure prediction via graph representation of error codes in accordance with one or more embodiments described herein.

FIGS. 4-5 illustrate example, non-limiting block diagrams regarding an order-of-receipt knowledge graph sequence, a retrospective time span, and a sliding time window in accordance with one or more embodiments described herein.

FIG. 6 illustrates a block diagram of an example, non-limiting system including a deep learning neural network and a scanner maintenance prediction that facilitates failure prediction via graph representation of error codes in accordance with one or more embodiments described herein.

FIGS. 7-11 illustrate example, non-limiting block diagrams showing how a deep learning neural network can generate a scanner maintenance prediction based on an order-of-receipt knowledge graph sequence in accordance with one or more embodiments described herein.

FIG. 12 illustrates a block diagram of an example, non-limiting system including a cyclicity diagram that facilitates failure prediction via graph representation of error codes in accordance with one or more embodiments described herein.

FIG. 13 illustrates an example, non-limiting illustration of a cyclicity diagram in accordance with one or more embodiments described herein.

FIG. 14 illustrates an example, non-limiting block diagram showing how a deep learning neural network can be trained in accordance with one or more embodiments described herein.

FIG. 15 illustrates a flow diagram of an example, non-limiting computer-implemented method that facilitates failure prediction via graph representation of error codes in accordance with one or more embodiments described herein.

FIG. 16 illustrates a block diagram of an example, non-limiting operating environment in which one or more embodiments described herein can be facilitated.

FIG. 17 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

A medical imaging scanner (e.g., a computed tomography (CT) scanner, a magnetic resonance imaging (MRI) scanner, an X-ray scanner, an ultrasound scanner, a positron emission tomography (PET) scanner, a nuclear medicine (NM) scanner, an image-guided surgery (IGS) machine) can electronically capture or generate a medical image (e.g., an array of pixels or voxels) that depicts or illustrates an anatomical structure (e.g., an organ, a tissue, a bodily fluid, a bodily cavity) of a medical patient (e.g., human, animal, or otherwise). In order for the medical image to be high quality (e.g., to have proper visual characteristics like brightness or contrast; to have no or few visual artifacts; to show the undistorted or otherwise true physical appearance of the anatomical structure), the medical imaging scanner should be in proper working condition (e.g., should be undamaged, properly serviced, or otherwise not in a state of disrepair). In situations where the medical imaging scanner suffers an operational failure (e.g., a hardware or software malfunction) and thus is no longer in proper working condition, the medical image can be degraded or possibly not generated at all. In such cases, diagnosis, prognosis, treatment, or therapy of the medical patient can suffer.

For example, suppose that the medical imaging scanner is an IGS machine. The IGS machine can provide to a surgeon a real-time view or visual feed (e.g., a timeseries of medical images) showing the medical patient's inner anatomy as the surgeon performs a surgical procedure on the medical patient. Suppose that the IGS machine were to malfunction or fail mid-surgery. In such situation, the surgeon can be unable to safely continue the surgical procedure or to safely remove or extricate their surgical equipment (e.g., scalpels, picks, tubes) from the medical patient. After all, the surgical procedure can be performed in a minimally-invasive fashion (e.g., where the surgeon inserts their surgical equipment through a small incision on the medical patient), such that the surgeon cannot see inside of the medical patient's body without the aid of the IGS machine. So, failure of the IGS machine during the surgical procedure can result in severe discomfort or injury for the medical patient.

Even in situations where the medical imaging scanner fails but not during or while being used on the medical patient, such failure can nevertheless be highly problematic. Indeed, such failure can be considered as unplanned downtime, and the opportunity cost of such unplanned downtime can be extremely large to whichever hospital or medical facility possesses the medical imaging scanner (e.g., a single day of unplanned downtime for a single scanner can correspond to an average loss or deferment of 5 planned scans, which can be equivalent to an average loss or deferment of tens of thousands of dollars in medical service revenue).

Accordingly, proactively preventing operational failures of the medical imaging scanner before they occur can be better than reactively responding to such failures after they occur. After all, reactive responses cause nearly all downtime to be unplanned and risk failures occurring during use or surgery. In contrast, proactive prevention can help to minimize or avoid unplanned downtime altogether, thereby reducing or eliminating the risk of failures occurring during use or surgery.

Existing techniques facilitate such proactive prevention by leveraging artificial intelligence. Specifically, many constituent pieces of hardware (e.g., X-ray tube filament, X-ray tube control board, gantry motor, gantry control board) that make up the medical imaging scanner can be equipped, outfitted, or otherwise instrumented with respective sensors (e.g., voltage sensors, current sensors, temperature sensors, pressure sensors, moisture sensors, deflection sensors). Accordingly, each instrumented piece of constituent hardware can produce one or more error codes, each error code being a distinct or unique electronic alert having a respective alphanumeric identifier (e.g., error code 3602011) or textual description (e.g., “out of range voltage”; “unable to retrieve queried data”; “unexpected event received in state machine”; “unknown error”) and being triggered in response to a respective combination of measurements captured by whatever sensors monitor that instrumented piece of constituent hardware. Note that, in any given time span (e.g., 60-day time span), multiple error codes can be produced by the medical imaging scanner. Existing techniques train an artificial intelligence model to receive as input a concatenation of whatever error codes are produced during that given time span and to generate as output some maintenance prediction associated with the medical imaging scanner (e.g., to predict a remaining useful life for some instrumented piece of constituent hardware; to recommend a specific maintenance task that should be performed on that instrumented piece of constituent hardware). In other words, the artificial intelligence model can be considered as learning how to detect or recognize within those inputted error codes subtle signatures that uniquely signal the identity of an upcoming or impending operational failure of the medical imaging scanner, and the maintenance prediction can be considered as providing specific direction or instruction regarding how to proactively avoid such upcoming or impending operational failure.

Although existing techniques that facilitate failure prediction for medical imaging scanners can achieve good accuracy, the inventors of various embodiments described herein realized that such existing techniques still leave room for improvement. Accordingly, the present inventors devised at least two specific optimizations to such existing techniques that allow or cause failure prediction accuracy to be heightened or boosted.

A first optimization devised by the present inventors relates to the format in which the inputted error codes are fed to the artificial intelligence model. Specifically, rather than feeding as input to the artificial intelligence model a concatenation of whatever error codes are generated during the given time span, various embodiments described herein can instead feed as input to the artificial intelligence model one or more knowledge graphs whose nodes respectively correspond to those error codes and whose edges indicate the order of receipt during the given time span of respective pairs of those error codes. In particular, the given time span can be broken up into a sequence of time intervals, such as via a sliding window that is incrementally scrolled or scrubbed across the given time span (e.g., a 60-day time span can be broken up into 46 distinct 15-day time intervals by scrolling or scrubbing a 15-day sliding window across that 60-day time span in 1-day increments). For each time interval, a respective knowledge graph can be constructed, such that each node of that knowledge graph represents a respective error code that could potentially be generated by the medical imaging scanner, and such that each edge of that knowledge graph is a directed relation representing the order of receipt between two respective error codes that were produced during that time interval (e.g., an edge traveling from an error code A to an error code B can indicate that the error code A was received or produced during that time interval before the error code B was received or produced). Accordingly, the nodes of that knowledge graph can be considered as representing the full set of error codes that could possibly be produced by the medical imaging scanner, and the edges of that knowledge graph can be considered as tracing out a contiguous or unbroken path through that full set of possible error codes, such contiguous or unbroken path showing or being the chronological order of whichever subset of that full set of possible error codes were actually produced during that time interval. In some situations, nodes representing error codes that were not produced during that time interval can be omitted from that knowledge graph (e.g., whichever nodes of that knowledge graph that are not coupled to any edges can be removed from that knowledge graph). In any case, by performing such graph construction for each of the sequence of time intervals, a sequence of knowledge graphs can be obtained (e.g., one respective knowledge graph per time interval). As described herein, the artificial intelligence model can be configured to receive as input that sequence of knowledge graphs, as opposed to a mere concatenation of the error codes that are generated during the given time span.

In devising this first optimization, the present inventors drew inspiration from social network theory. Social network theory is a field of study that examines how large numbers of atomized or independent social actors (e.g., individuals, companies) interact with each other over time. A social network can be structured as a graph whose nodes represent respective social actors and whose edges represent communications (sometimes referred to as “tweets”), with each communication originating from one respective social actor and being received by another respective social actor. Some social actors can be central or influential in the social network (e.g., can send or receive many communications), whereas other social actors can be peripheral or non-influential in the social network (e.g., can send or receive few communications). By formatting the social network as such a graph, sociologically complex or emergent communication behaviors (e.g., cascade effects, reputational effects, echo chambers, social contagions) can be more easily revealed or recognized than if the social network were instead formatted differently (e.g., were instead formatted as a mere list of communications).

The present inventors realized that the principles of social network theory can be utilized to increase the accuracy of failure prediction for the medical imaging scanner. Indeed, by formatting the error codes received during the given time span as the above-mentioned sequence of knowledge graphs rather than as a mere concatenation or list, it can be easier for the artificial intelligence model to detect or recognize the subtle signatures of upcoming or impending scanner failures that are contained within or signaled by the error codes. In other words, the present inventors realized that a significant portion of such subtle signatures can be contained within the temporality or chronology in which such error codes are received, as opposed to merely being contained within the explicit content of the individual error codes themselves, and the present inventors further realized that such temporality or chronology can be more easily revealed to or gleaned by the artificial intelligence model when the error codes are formatted as the above-mentioned sequence of knowledge graphs rather than when the error codes are formatted as a mere timestamped list. In still other words, the present inventors realized that, just as graph formatting allows sociologically complex or emergent communication behaviors to be more easily recognized in social networks, so too can graph formatting allow upcoming or impending operational failures to be more easily recognized in the error codes of medical imaging scanners.

However, the present inventors realized that successfully applying the principles of social network theory to the medical imaging scanner is not straightforward. After all, in a social network graph, the nodes represent social actors that send communications to each other. So, a straightforward application of the principles of social network theory to the medical imaging scanner would involve constructing knowledge graphs whose nodes represent entities that send communications to each other. In the medical imaging scanner, the only entities that send communications are the instrumented pieces of constituent hardware that make up the medical imaging scanner, their communications being their respective error codes. However, those instrumented pieces of constituent hardware do not send error codes to each other (e.g., a gantry motor does not send an error code to an X-ray tube). Instead, those instrumented pieces of constituent hardware all send their error codes to a centralized controller of the medical imaging scanner. If a knowledge graph were constructed in straightforward fashion without a node representing that centralized controller, there would be no edges whatsoever in that knowledge graph. Thus, such knowledge graph would be utterly useless. On the other hand, if a knowledge graph were constructed in straightforward fashion with a node representing that centralized controller, every single edge in that knowledge graph would lead toward that node, such that there would be no contiguous or unbroken path traced throughout that knowledge graph. Although such knowledge graph might not be utterly useless, the present inventors realized that its lack of a contiguous or unbroken path would obfuscate or make murkier the temporality or chronology in which the error codes are received, meaning that it would not be easier for the artificial intelligence model to recognize the subtle signature of an upcoming or impending scanner failure from such knowledge graph as compared to a mere concatenation or list of timestamped error codes.

Fortunately, the present inventors devised an innovative tweak or modification that enables the principles of social network theory to be successfully leveraged so as to increase the accuracy of failure prediction for the medical imaging scanner. Specifically, rather than constructing knowledge graphs whose nodes are instrumented pieces of constituent hardware and whose edges are error codes transmitted by those instrumented pieces of constituent hardware, various embodiments described herein instead construct knowledge graphs whose nodes are the error codes themselves and whose edges indicate relative order of receipt or arrival of those error codes. In other words, the innovative tweak or modification concocted by the present inventors can be to respectively replace: the actors and actor-transmitted communications of social network theory; with actor-transmitted communications and their relative order of receipt or arrival. By implementing this innovative tweak or modification with respect to the medical imaging scanner, each knowledge graph constructed for the medical imaging scanner can exhibit or have its own contiguous or unbroken path traced through it, which can more easily reveal to the artificial intelligence model signatures of any upcoming or impending scanner failure.

A second optimization devised by the present inventors relates to the inclusion of not just the temporality or chronology of error codes produced by the medical imaging scanner, but also to the inclusion of spatiality of such error codes. Specifically, various embodiments described herein can involve constructing or configuring the artificial intelligence model such that it contains a convolutional layer which convolves: each of the sequence of knowledge graphs; with an error-code-to-hardware mapping kernel that indicates, for each unique error code that is producible by the medical imaging scanner, which respective instrumented piece of constituent hardware is configured to generate that error code. In other words, the error-code-to-hardware mapping kernel can be considered as a mapping between error codes and instrumented pieces of constituent hardware, and convolving each of the sequence of knowledge graphs with that error-code-to-hardware mapping kernel can cause each knowledge graph to represent: not just the chronological order in which error codes are received during a respective time interval; but also the physical pieces of hardware that transmitted those error codes during that time interval. In other words, the sequence of knowledge graphs can initially be considered as being in a temporal domain (e.g., as only representing the temporality or chronology of error codes), and the sequence of knowledge graphs after such convolution can be considered as instead being in a spatio-temporal domain (e.g., as representing both the temporality or chronology of error codes and the spatial locations on or in the medical imaging scanner from which respective error codes originate). As mentioned above, the nodes of the sequence of knowledge graphs can indicate actor-transmitted communications (e.g., error codes) rather than actors (e.g., instrumented pieces of constituent hardware), and the edges of the sequence of knowledge graphs can indicate the relative order or chronology in which such actor-transmitted communications are received or produced. Thus, before such convolution, the sequence of knowledge graphs can be considered as conveying no information about the actual actors (e.g., instrumented pieces of constituent hardware). However, after such convolution, the sequence of knowledge graphs can be considered as now conveying information about the actual actors, not just the actor-transmitted communications and their order of receipt. Additionally, such convolution allows the sequence of knowledge graphs to convey such actor information without suffering from the above-mentioned absence of contiguous or unbroken paths traced through each knowledge graph.

In any case, by implementing the various optimizations described herein (e.g., formatting error codes as order-of-receipt knowledge graphs rather than mere concatenations or lists; convolving such order-of-receipt knowledge graphs with a mapping between error codes and the specific instrumented hardware that generates those error codes), failures of the medical imaging scanner can be more accurately, reliably, or robustly predicted in advance, thereby helping to reduce unplanned downtime or situations in which the medical imaging scanner fails mid-surgery.

Various embodiments described herein can be employed to use hardware or software to solve problems that are highly technical in nature (e.g., to facilitate failure prediction via graph representation of error codes), that are not abstract and that cannot be performed as a set of mental acts by a human. Further, some of the processes performed can be performed by a specialized computer (e.g., deep learning neural network) for carrying out defined acts related to scanner failure prediction. Such defined acts can include accessing, by a device operatively coupled to a processor, a medical imaging scanner, wherein: the medical imaging scanner has a plurality of instrumented hardware parts; each of the plurality of instrumented hardware parts is monitored by a respective set of physical sensors; and each of the plurality of instrumented hardware parts is configured to produce one or more respective electronic error codes in response to one or more respective combinations of measurements from its physical sensors; constructing, by the device, a sequence of order-of-receipt knowledge graphs based on monitoring the plurality of instrumented hardware parts over a sequence of time intervals, wherein: a first order-of-receipt knowledge graph of the sequence of order-of-receipt knowledge graphs corresponds to a first time interval of the sequence of time intervals; each node of the first order-of-receipt knowledge graph represents a respective electronic error code producible by the plurality of instrumented hardware parts; and each edge of the first order-of-receipt knowledge graph represents an order in which two respective electronic error codes are produced by the plurality of instrumented hardware parts during the first time interval; and estimating or recommending, by the device, a remaining useful life of or a maintenance task for at least one of the plurality of instrumented hardware parts, based on executing a deep learning neural network on the sequence of order-of-receipt knowledge graphs. In various aspects, the deep learning neural network can convolve each of the sequence of order-of-receipt knowledge graphs with a kernel indicating to which of the plurality of instrumented hardware parts respective error codes belong.

Such defined acts are inherently computerized. Indeed, medical imaging scanners (e.g., X-ray scanners, MRI scanners, CT scanners) are specific computerized hardware. Neither such scanners nor the images that they capture can be implemented in the human mind or with mere pen and paper. Additionally, the field of medical imaging scanner failure prediction focuses on forecasting when or what specific types of service, repair, or maintenance tasks should be performed in advance on medical imaging scanners so as to avoid operational malfunctions (e.g., hardware damage, software glitches). It would make no sense whatsoever to contend that the proactive service, repair, or maintenance of a medical imaging scanner could somehow be performed wholly in the human mind or on just a piece of paper. Additionally, artificial intelligence models (e.g., deep learning neural networks) are also inherently computerized constructs made up of specific software-oriented architectures (e.g., input layers, hidden layers, or output layers, any of which can be made up of trainable or non-trainable internal parameters such as convolutional layers or long short-term memory (LSTM) layers). Artificial intelligence models cannot be trained or executed by the human mind, or by humans with mere pen and paper, in any reasonable or practicable way without computers.

Moreover, various embodiments described herein can integrate into a practical application various teachings relating to failure prediction of medical imaging scanners. As described above, reactively responding to operational failures of medical imaging scanners results in significant unplanned downtime and risks failures occurring mid-surgery. To avoid these issues, proactive prediction of operational failures of medical imaging scanners can be facilitated via artificial intelligence. Existing techniques achieve such proactive prediction by concatenating together all the error codes produced by a medical imaging scanner during a given time span and feeding that concatenation to a trained machine learning model which generates as output a maintenance prediction for the medical imaging scanner. Although such existing techniques achieve good results, the present inventors realized that there are nevertheless various opportunities for improvement or optimization.

Specifically, the present inventors realized that a higher level of failure prediction accuracy can be achieved by changing the format according to which the machine learning model receives the error codes of the medical imaging scanner as input. In particular, rather than receiving a concatenation or list of error codes as input, the machine learning model can instead be configured to receive as input a sequence of knowledge graphs whose nodes represent distinct error codes and whose edges represent relative order or chronology of receipt of those error codes. Drawing inspiration from the field of social network theory, the present inventors realized that such a graph format of the error codes can make it easier for the machine learning model to recognize or detect whatever subtle signatures, precursors, or signalers of upcoming scanner failures are hidden throughout those error codes. Indeed, such a graph format can be considered as better revealing complex or emergent patterns that are contained within the temporality or chronology of the error codes, as compared to instead formatting those error codes as a mere concatenation or list. Furthermore, the present inventors realized that spatial information can also be taken into account, by convolving each of the sequence of knowledge graphs with a mapping that shows which error codes are produced or producible by which specific pieces of instrumented hardware of the medical imaging scanner. In such situations, the sequence of knowledge graphs can, after such convolutions, be considered as highlighting or emphasizing both temporal and spatial interrelationships among the error codes, thus making it easier for the machine learning model to discern such interrelationships. Without formatting or convolving the error codes as described herein, such temporal and spatial interrelationships would be much more obfuscated or hidden, thus making it more difficult for the machine learning model to discern them. In other words, existing techniques can be considered as trusting the model to find or recognize such temporal and spatial interrelationships for itself, whereas various embodiments described herein can be considered as an error code formatting tweak which allows the machine learning model to more easily or more readily find or recognize such temporal and spatial interrelationships than would otherwise be possible. In fact, experiments and simulations conducted by the present inventors on a population of 907 deployed medical imaging scanners over a 12-month period indicate that various embodiments described herein can achieve nearly a 36% reduction in unplanned downtime (e.g., can save about 18.5 hours of unplanned downtime) compared to existing techniques. Such experiments or simulations clearly demonstrate how various embodiments described herein can outperform existing techniques.

Furthermore, it must be emphasized how counterintuitive various embodiments described herein are. As mentioned above, the present inventors concocted various embodiments by drawing inspiration from social network theory. Social network theory is from the field of sociology, which is entirely unrelated to the field of failure prediction for medical imaging scanners. Accordingly, a person having ordinary skill in the art who desired to increase the accuracy of failure prediction for medical imaging scanners would not have even considered implementing the principles of social network theory. Such person of ordinary skill would have instead attempted to achieve such increase in failure prediction accuracy by collecting more training data for their machine learning model or by increasing the number or variety of sensors monitoring the constituent pieces of hardware that make up the medical imaging scanner.

Even if a person of ordinary skill entertained the idea of applying the principles of social network theory to the field of failure prediction for medical imaging scanners, they would have swiftly encountered significant roadblocks and, accordingly, would have abandoned such idea. After all, social network theory involves representing a social network as a graph whose nodes are social actors and whose edges are communications between social actors. The medical imaging scanner analogue of a social actor is an instrumented piece of constituent hardware, and the medical imaging scanner analogue of a communication produced by such an actor is an error code. Thus, a straightforward application of the principles of social network theory to the field of failure prediction for medical imaging scanners would involve constructing graphs whose nodes are instrumented pieces of constituent hardware and whose edges are error codes. But, as explained above, such a construction would not better reveal, highlight, or emphasize the temporal interrelationships between error codes, since instrumented pieces of constituent hardware do not transmit error codes to each other. Instead, all instrumented pieces of constituent hardware transmit their error codes to a centralized controller of the medical imaging scanner. If a straightforward knowledge graph had no node that represented such centralized controller, there would be no edges whatsoever in that straightforward knowledge graph, which would render it completely useless. On the other hand, if that straightforward knowledge graph had a node that represented such centralized controller, every single edge in that straightforward knowledge graph would point toward that node. In such case, failure prediction accuracy would not be boosted since, as the present inventors recognized, the temporality or chronology between error codes would be obfuscated within such straightforward knowledge graph. A person of ordinary skill would thus not achieve any performance boost and would accordingly abandon any attempt to leverage the principles of social network theory in the context of failure prediction for medical imaging scanners.

In stark contrast, the present inventors devised an innovative way to apply the principles of social network theory to the field of failure prediction for medical imaging scanners. Rather than constructing graphs whose nodes represent instrumented pieces of constituent hardware and whose edges represent error codes, various embodiments can instead construct graphs whose nodes represent error codes and whose edges represent the relative order of receipt or arrival of such error codes. In other words, rather than having nodes that represent actors and edges that represent communication between actors, various embodiments can instead have nodes that represent communications and edges that represent chronological order between communications. As the present inventors'experiments and simulations show, constructing a graph in such fashion can be considered as emphasizing or revealing temporal interrelationships between error codes which would have otherwise been hidden or obfuscated, such that it can be easier for a machine learning model to predict scanner failures. Although a graph whose nodes represent error codes rather than instrumented pieces of constituent hardware can be considered as lacking or hiding spatial interrelationships between such error codes, those spatial interrelationships can be reinserted or unhidden by convolving such graph with a kernel that indicates or maps which error codes are produced by which instrumented pieces of constituent hardware. Thus, various embodiments described herein can be considered as a clever, strange, or unusual way of formatting error codes so as to emphasize or highlight their temporal and spatial interrelationships, thereby boosting failure predictions made from such error codes.

For at least these reasons, various embodiments can address or ameliorate technical problems or disadvantages that afflict existing techniques. Therefore, such embodiments constitute a concrete and tangible technical improvement or technical effect in the field of failure prediction for medical imaging scanners. Accordingly, such embodiments certainly qualify as useful and practical applications of computers.

It should be appreciated that the herein figures and description provide non-limiting examples of various embodiments and are not necessarily drawn to scale.

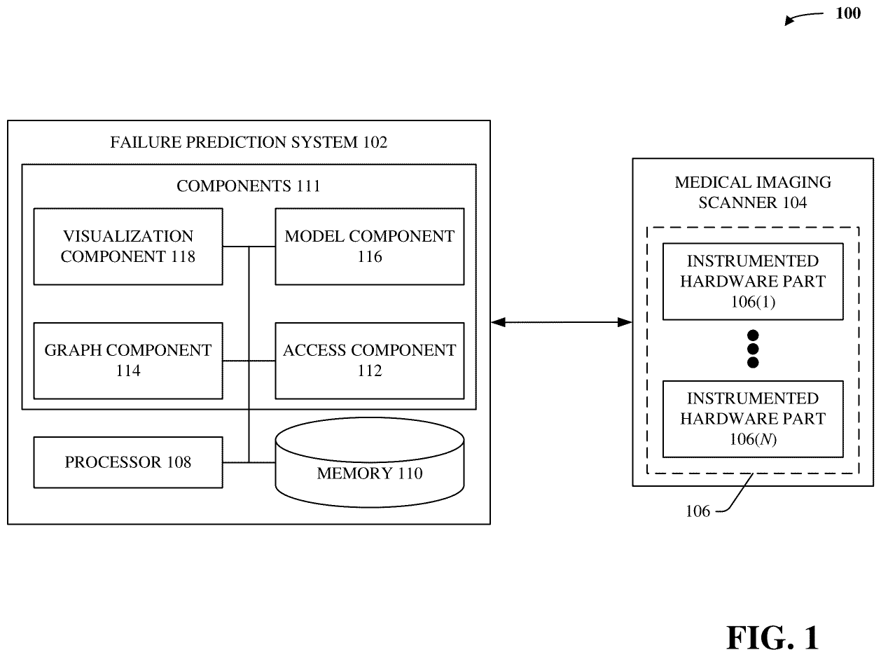

FIG. 1 illustrates a block diagram of an example, non-limiting system 100 that can facilitate failure prediction via graph representation of error codes in accordance with one or more embodiments described herein. As shown, a failure prediction system 102 can be electronically integrated, via any suitable wired or wireless electronic connections, with a medical imaging scanner 104.

In various embodiments, the medical imaging scanner 104 can be any suitable type of equipment or modality that can electronically capture or otherwise electronically generate images (e.g., two-dimensional arrays of pixels, each pixel exhibiting a respective Hounsfield unit value; or three-dimensional arrays of voxels, each voxel exhibiting a respective Hounsfield unit value) that depict or illustrate any suitable anatomical structures (e.g., bodily organs or portions thereof, bodily tissues or portions thereof, bodily cavities or portions thereof, bodily fluids or portions thereof, bodily implants or portions thereof, bodily prostheses or portions thereof) of medical patients (e.g., human patients, animal patients, other types of patients). As a non-limiting example, the medical imaging scanner 104 can be an X-ray scanner, in which case the medical imaging scanner 104 can capture or generate scanned X-ray images of medical patients for purposes of diagnosing or prognosing those medical patients. As another non-limiting example, the medical imaging scanner 104 can be a CT scanner, in which case the medical imaging scanner 104 can capture or generate scanned CT images of medical patients for purposes of diagnosing or prognosing those medical patients. As yet another non-limiting example, the medical imaging scanner 104 can be an MRI scanner, in which case the medical imaging scanner 104 can capture or generate scanned MRI images of medical patients for purposes of diagnosing or prognosing those medical patients. As even another non-limiting example, the medical imaging scanner 104 can be an ultrasound scanner, in which case the medical imaging scanner 104 can capture or generate scanned ultrasound images of medical patients for purposes of diagnosing or prognosing those medical patients. As still another non-limiting example, the medical imaging scanner 104 can be a PET scanner, in which case the medical imaging scanner 104 can capture or generate scanned PET images of medical patients for purposes of diagnosing or prognosing those medical patients. As another non-limiting example, the medical imaging scanner 104 can be an NM scanner, in which case the medical imaging scanner 104 can capture or generate scanned NM images of medical patients for purposes of diagnosing or prognosing those medical patients. As yet another non-limiting example, the medical imaging scanner 104 can be an IGS machine, in which the medical imaging scanner 104 can capture any of the above-mentioned types of images of medical patients, as well as any visible-spectrum images of those medical patients, for purposes of planning surgeries on those medical patients or navigating during the surgeries of those medical patients.

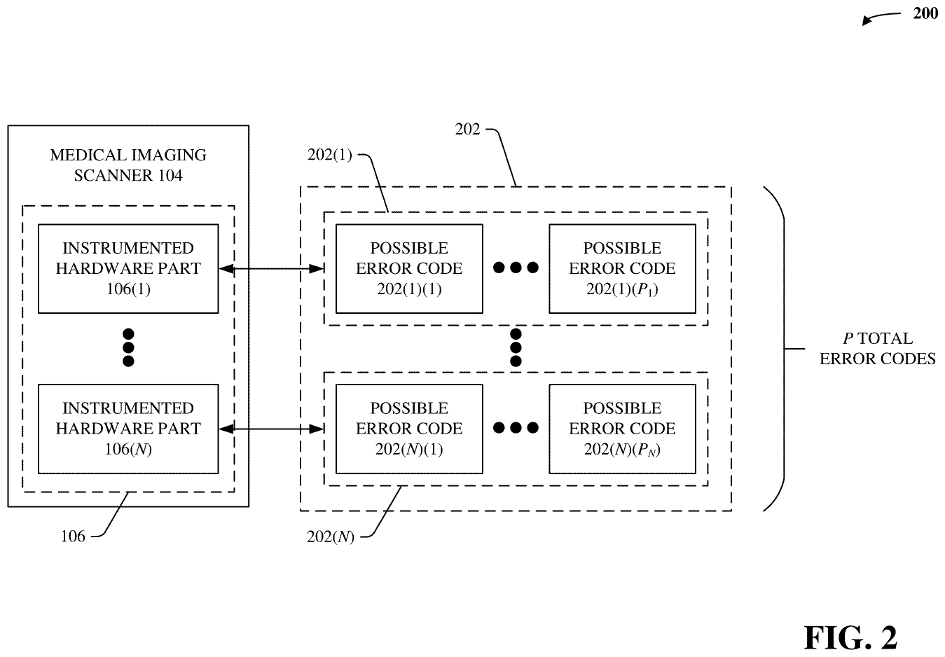

In various embodiments, the medical imaging scanner 104 can have or otherwise be made up of a plurality of instrumented hardware parts 106. The plurality of instrumented hardware parts 106 can include a total of n parts, for any suitable positive integer n>1: an instrumented hardware part 106(1) to an instrumented hardware part 106(n). Each of the plurality of instrumented hardware parts 106 can be any suitable distinct or unique piece of physical equipment, machinery, or gadgetry that is a constituent piece of the medical imaging scanner 104 and that the medical imaging scanner 104 utilizes in some way to capture, record, or generate medical images. As some non-limiting examples, any of the plurality of instrumented hardware parts 106 can be: an X-ray tube; an X-ray tube chiller; an X-ray tube inverter; an X-ray tube control printed circuit board (PCB); a high-voltage tank; a gantry motor; a gantry control PCB; a table motor; a table control PCB; a collimator; an optical filter or lens; an image detector; an image detector control PCB; a graphical user interface (GUI); a GUI control PCB; or a footswitch. In any case, each of the plurality of instrumented hardware parts can be outfitted with or otherwise monitored by one or more respective sensors, hence the term “instrumented.” As some non-limiting examples, any of the plurality of instrumented hardware parts 106 can be outfitted with or otherwise monitored by: an electric voltage sensor; an electric current sensor; an electric resistance sensor; a temperature sensor; a pressure sensor; a moisture sensor; a strain or deflection sensor; an accelerometer; a gyroscope; a vibration sensor; or a sound sensor. In other words, the physical state (e.g., in terms of voltage, current, resistance, temperature, pressure, moisture, strain, deflection, acceleration, spatial orientation, vibration, or sound) of each of the plurality of instrumented hardware parts 106 can be continuously, continually, periodically, or aperiodically monitored or surveilled by its respective sensors. In should be understood or otherwise appreciated that any two of the plurality of instrumented hardware parts 106 can be monitored by the same or different numbers or types of sensors as each other. In any case, each of the plurality of instrumented hardware parts 106 can be configured to electronically generate or transmit various error codes in response to various respective combinations of measurements from its outfitted sensors. Non-limiting aspects are described with respect to FIG. 2.

FIG. 2 illustrates an example, non-limiting block diagram 200 of the plurality of instrumented hardware parts 106 in accordance with one or more embodiments described herein. As shown, the plurality of instrumented hardware parts 106 can respectively correspond with a plurality of sets of possible error codes 202.

As a non-limiting example, the instrumented hardware part 106(1) can correspond to a set of possible error codes 202(1), which can include a total of p₁ codes for any suitable positive integer p₁: a possible error code 202(1)(1) to a possible error code 202(1)(p₁). The possible error code 202(1)(1) can be any suitable electronic alert having a unique or distinct alphanumeric identifier or name, having a unique or distinct textual description of its significance or otherwise of what type or sort of technical issue that it represents, and being triggered or produced in response to some first combination of measurement readouts being recorded by whatever sensors with which the instrumented hardware part 106(1) is equipped. Likewise, the possible error code 202(1)(p₁) can be any suitable electronic alert having a unique or distinct alphanumeric identifier or name, having a unique or distinct textual description of its significance or otherwise of what type or sort of technical issue that it represents, and being triggered or produced in response to some p₁-th combination of measurement readouts being recorded by whatever sensors with which the instrumented hardware part 106(1) is equipped. In other words, the instrumented hardware part 106(1) can be considered as having p₁ distinct options of error codes to produce during any given time-step or moment.

As another non-limiting example, the instrumented hardware part 106(n) can correspond to a set of possible error codes 202(n), which can include a total of pₙ codes for any suitable positive integer pₙ: a possible error code 202(n)(1) to a possible error code 202(n)(pₙ). The possible error code 202(n)(1) can be any suitable electronic alert having a unique or distinct alphanumeric identifier or name, having a unique or distinct textual description of its significance or otherwise of what type or sort of technical issue that it represents, and being triggered or produced in response to some first combination of measurement readouts being recorded by whatever sensors with which the instrumented hardware part 106(n) is equipped. Similarly, the possible error code 202(n)(pₙ) can be any suitable electronic alert having a unique or distinct alphanumeric identifier or name, having a unique or distinct textual description of its significance or otherwise of what type or sort of technical issue that it represents, and being triggered or produced in response to some pₙ-th combination of measurement readouts being recorded by whatever sensors with which the instrumented hardware part 106(n) is equipped. That is, the instrumented hardware part 106(n) can be considered as having pₙ distinct options of error codes to produce during any given time-step or moment.

Note that the set of possible error codes 202(1) to the set of possible error codes 202(n) can collectively be considered as forming the plurality of sets of possible error codes 202.

Additionally, note that the total cardinality of the plurality of sets of possible error codes 202 can be p. In other words, it can be the case that

$${\sum }_{i = 1}^{n} {p}_{i} = p .$$

Referring back to FIG. 1, it can be desired to proactively predict when or what types of operational failures the medical imaging scanner 104 is likely to experience in the future, so that such operational failures can be preemptively repaired or avoided. As described herein, the failure prediction system 102 can facilitate such proactive prediction.

In various embodiments, the failure prediction system 102 can include a processor 108 (e.g., computer processing unit, microprocessor) and a non-transitory computer-readable memory 110 that is operably or operatively or communicatively connected or coupled to the processor 108. The non-transitory computer-readable memory 110 can store computer-executable instructions which, upon execution by the processor 108, can cause the processor 108 or other components of the failure prediction system 102 (e.g., access component 112, graph component 114, model component 116, visualization component 118) to perform one or more acts. In various embodiments, the non-transitory computer-readable memory 110 can store computer-executable components (e.g., access component 112, graph component 114, model component 116, visualization component 118), and the processor 108 can execute the computer-executable components.

In various embodiments, the failure prediction system 102 can include an access component 112. In various aspects, the access component 112 can electronically communicate with, can electronically control, or can otherwise electronically access the medical imaging scanner 104. Accordingly, the access component 112 can electronically transmit any suitable electronic data to, or receive any suitable electronic data from, the medical imaging scanner 104. In some cases, the access component 112 can be considered as a proxy or conduit by which other components of the failure prediction system 102 can electronically interact with the medical imaging scanner 104.

In various embodiments, the failure prediction system 102 can include a graph component 114. In various aspects, the graph component 114 can, as described herein, electronically construct a sequence of order-of-receipt knowledge graphs by monitoring error codes produced during a retrospective time span that is measured backwards from a current time or date.

In various embodiments, the failure prediction system 102 can include a model component 116. In various instances, the model component 116 can, as described herein, electronically generate a scanner maintenance prediction, based on executing a deep learning neural network on the sequence of order-of-receipt knowledge graphs.

In various embodiments, the failure prediction system 102 can include a visualization component 118. In various cases, the visualization component 118 can, as described herein, render or display a cyclicity diagram, which can depict a visual order-of-receipt error code pattern that signifies or is a signature of whatever operational failure is indicated by the scanner maintenance prediction.

Note that the access component 112, the graph component 114, the model component 116, and the visualization component 118 can collectively be considered as being one or more components 111 of the failure prediction system 102. It should be appreciated that the one or more components 111 are described primarily herein as comprising four components for ease of explanation and illustration. However, the one or more components 111 are not limited to being implemented as exactly such four components in every embodiment. Indeed, in some embodiments, the functionalities described herein of such four components can be combined in any suitable fashions, so as to be implemented in or by fewer than four components (e.g., in some cases, a single component can perform all of the functionalities that are described herein with respect to the access component 112, the graph component 114, the model component 116, and the visualization component 118). In other embodiments, the functionalities described herein of such four components can instead be distributed, separated, split, or fragmented in any suitable fashions, so as to be implemented in or by more than four components (e.g., two or more components can facilitate the functionalities that are performable by the access component 112; two or more components can facilitate the functionalities that are performable by the graph component 114; two or more components can facilitate the functionalities that are performable by the model component 116; two or more components can facilitate the functionalities that are performable by the visualization component 118).

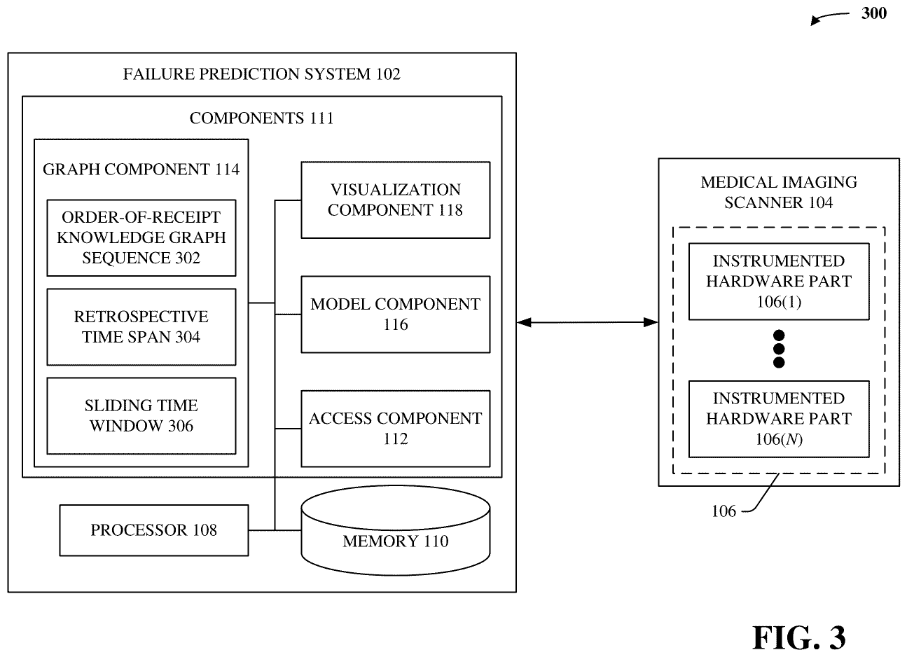

FIG. 3 illustrates a block diagram of an example, non-limiting system 300 including an order-of-receipt knowledge graph sequence, a retrospective time span, and a sliding time window that can facilitate failure prediction via graph representation of error codes in accordance with one or more embodiments described herein. As shown, the system 300 can include the same components as the system 100, and can further include an order-of-receipt knowledge graph sequence 302, a retrospective time span 304, or a sliding time window 306.

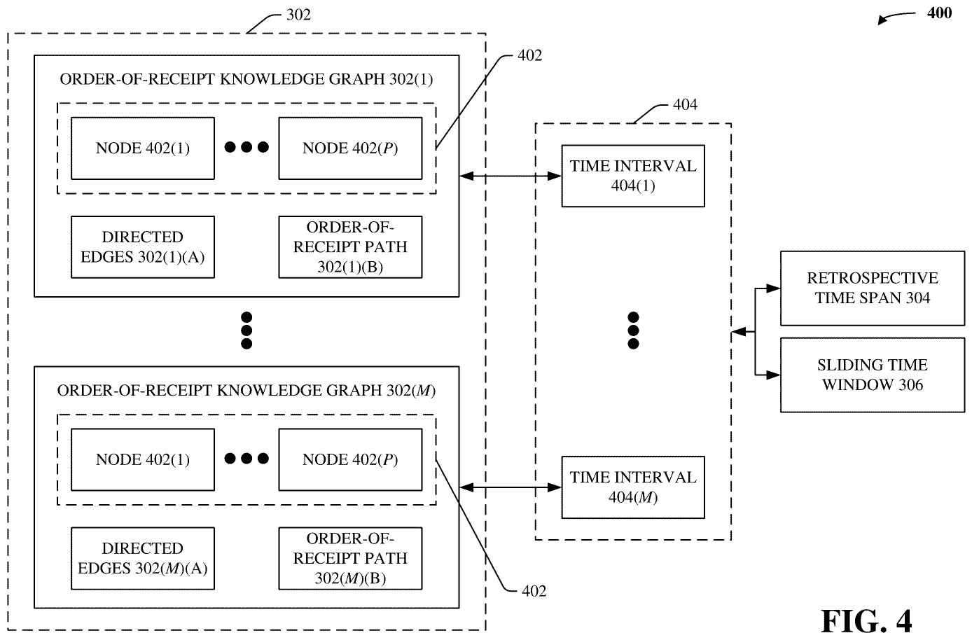

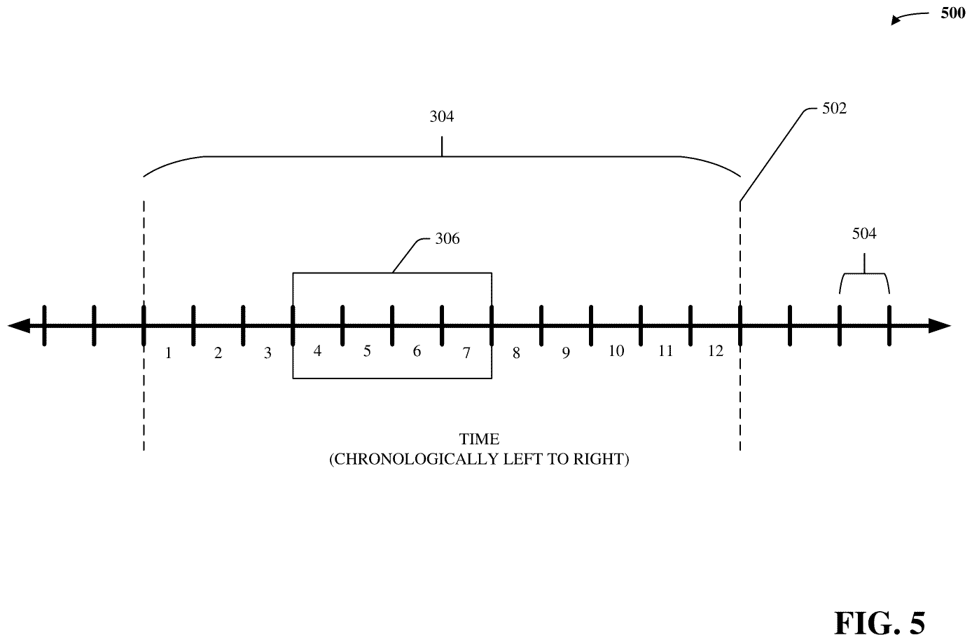

In various embodiments, the retrospective time span 304 can be any suitable length of time that is measured backwards from a current time or date, hence the term “retrospective.” In contrast, the sliding time window 306 can be any suitable length of time which is shorter than the retrospective time span 304 and, thus, which can be slid, scrolled, or scrubbed across the retrospective time span 304 (e.g., in an early-to-later direction) by defined temporal increments. In various aspects, the graph component 114 can electronically break-up the retrospective time span 304 into a sequence of time intervals using the sliding time window 306, and the graph component 114 can electronically construct the order-of-receipt knowledge graph sequence 302 by monitoring, logging, or otherwise examining whichever error codes are or were produced by the plurality of instrumented hardware parts 106 during such sequence of time intervals. Non-limiting aspects are described with respect to FIGS. 4-5.

FIGS. 4-5 illustrate example, non-limiting block diagrams 400 and 500 regarding the order-of-receipt knowledge graph sequence 302 in accordance with one or more embodiments described herein.

The graph component 114 can identify or otherwise create a sequence of time intervals 404, based on scrubbing or shifting the sliding time window 306 across the retrospective time span 304. The sequence of time intervals 404 can include a total of m intervals, for any suitable positive integer m>1: a time interval 404(1) to a time interval 404(m). Each of the sequence of time intervals 404 can be considered as the result of: scrubbing or shifting the sliding time window 306 to a respective location along the retrospective time span 304; and truncating away all portions of the retrospective time span 304 that fall outside of the sliding time window 306 when scrubbed or shifted to that respective location. As a non-limiting example, the time interval 404(1) can be obtained or delineated by: scrubbing or shifting the sliding time window 306 to some first location along the retrospective time span 304; and omitting all portions of retrospective time span 304 that are not contained within the sliding time window 306 when it is positioned at that first location. As another non-limiting example, the time interval 404(m) can be obtained or delineated by: scrubbing or shifting the sliding time window 306 to some m-th location along the retrospective time span 304; and omitting all portions of retrospective time span 304 that are not contained within the sliding time window 306 when it is positioned at that m-th location. Note that the chronological order of the retrospective time span 304 can be maintained or preserved in the sequence of time intervals 404. That is, for any suitable positive integer i<m, it can be the case that a time interval 404(i) precedes or otherwise begins before a time interval 404(i+1). In other words, the i-th position to which the sliding time window 306 is shifted along the retrospective time span 304 can be nearer to an earliest end (or, equivalently, can be farther from a latest end) of the retrospective time span 304 than can the (i+1)-th position to which the sliding time window 306 is shifted. In some situations, the sequence of time intervals 404 can be disjoint. That is, no two of the sequence of time intervals 404 can overlap with each other. In other situations, the sequence of time intervals 404 can be non-disjoint. That is, two or more of the sequence of time intervals 404 can overlap with each other. In any case, a union of the sequence of time intervals 404 can be equal to the retrospective time span 304.

FIG. 5 shows a non-limiting conceptual illustration of how the sliding time window 306 can be scrubbed or shifted along the retrospective time span 304. In particular, FIG. 5 depicts a timeline extending from left to right. For ease of explanation, the chronological flow of the timeline can thus be left to right, such that points that are more leftward on the timeline are temporally earlier than points that are more rightward on the timeline. As shown, the timeline can be spanned by equally-spaced tick marks, the spacing between any two adjacent tick marks representing some defined temporal increment 504. As some non-limiting examples, the defined temporal increment 504 can be one hour, one day, one week, or any other suitable amount of time. As shown, the retrospective time span 304 can be measured backwards (e.g., leftward since time flows chronologically left to right) from a current or present time 502. In the non-limiting example of FIG. 5, the length of the retrospective time span 304 is equal to 12 multiplied by the length of the defined temporal increment 504 (e.g., if the defined temporal increment 504 is one hour, then the retrospective time span 304 would, in this non-limiting example, be a 12-hour time span measured backwards from the current or present time 502; if the defined temporal increment 504 is one day, then the retrospective time span 304 would, in this non-limiting example, be a 12-day time span measured backwards from the current or present time 502). For ease of explanation, each of the twelve instantiations of the defined temporal increment 504 that make up the retrospective time span 304 are labeled 1 to 12 from earliest to latest. In any case, the sliding time window 306 can be shorter in length than the retrospective time span 304. In the non-limiting example of FIG. 5, the length of the sliding time window 306 is equal to 4 multiplied by the defined temporal increment 504. Accordingly, the sliding time window 306 can be scrubbed or shifted along the retrospective time span 304, so as to create the sequence of time intervals 404. As a non-limiting example, the sliding time window 306 can begin at an earliest (most leftward) end of the retrospective time span 304 and can be scrubbed or shifted later (rightward) by one instantiation of the defined time increment 504 per scrub or shift, until the sliding time window 306 reaches the latest (most rightward) end of the retrospective time span 304. This would yield a total of nine time intervals organized in sequence (e.g., a first of such nine time intervals would include labels 1 to 4; a second of such nine time intervals would include labels 2 to 5; a third of such nine time intervals would include labels 3 to 6; a fourth of such nine time intervals would include labels 4 to 7; a fifth of such nine time intervals would include labels 5 to 8; a sixth of such nine time intervals would include labels 6 to 9; a seventh of such nine time intervals would include labels 7 to 10; an eighth of such nine time intervals would include labels 8 to 11; and a ninth of such nine time intervals would include labels 9 to 12). As another non-limiting example, the sliding time window 306 can begin at an earliest (most leftward) end of the retrospective time span 304 and can be scrubbed or shifted later (rightward) by x instantiations of the defined time increment 504 per scrub or shift, until the sliding time window 306 reaches the latest (most rightward) end of the retrospective time span 304, where x can be equal to or otherwise based on the ratio of the length of the retrospective time span 304 to the length of the sliding time window 306. This would yield a total of three time intervals organized in sequence (e.g., a first of such three time intervals would include labels 1 to 4; a second of such three time intervals would include labels 5 to 8; and a third of such three time intervals would include labels 9 to 12). In any case more generally, the sequence of time intervals 404 can include a total of m intervals, where m can be dictated by the size of the retrospective time span 304, by the size of the sliding time window 306, and by how many instantiations of the defined temporal increment 504 are used to scrub or shift the sliding time window 306. Referring back to FIG. 4, the graph component 114 can construct, build, or otherwise generate the order-of-receipt knowledge graph sequence 302, based on whichever error codes are or were produced by the plurality of instrumented hardware parts 106 during respective ones of the sequence of time intervals 404.

As a non-limiting example, the graph component 114 can create an order-of-receipt knowledge graph 302(1) based on the time interval 404(1). The order-of-receipt knowledge graph 302(1) can be a digraph that is made up of nodes and directed edges.

More specifically, the order-of-receipt knowledge graph 302(1) can contain a plurality of nodes 402. The plurality of nodes 402 can include a total of p nodes: a node 402(1) to a node 402(p). In other words, the plurality of nodes 402 can respectively correspond (e.g., in one-to-one fashion) with the possible error codes that are producible by the plurality of instrumented hardware parts 106. In still other words, the plurality of nodes 402 can include one node per possible error code that the medical imaging scanner 104 is capable of producing. Accordingly, each of the plurality of nodes 402 can represent a respective possible error code from the plurality of sets of possible error codes 202. For instance, the node 402(1) can represent a first possible error code (e.g., 202(1)(1)) from the plurality of sets of possible error codes 202. So, the node 402(1) can carry a textual payload, where such textual payload can be one or more structured or unstructured text strings indicating the alphanumeric identifier or textual description of that first possible error code. As another instance, the node 402(p) can represent a p-th possible error code (e.g., 202(n)(pₙ)) from the plurality of sets of possible error codes 202. That is, the node 402(p) can carry a textual payload, where such textual payload can be one or more structured or unstructured text strings indicating the alphanumeric identifier or textual description of that p-th possible error code.

Moreover, the order-of-receipt knowledge graph 302 can contain a set of directed edges 302(1)(A), which can include any suitable number of directed edges. Each one of the set of directed edges 302(1)(A) can point or lead away from one respective node in the plurality of nodes 402 and toward another respective node in the plurality of nodes 402. In various aspects, each of the set of directed edges 302(1)(A) can represent a chronological order of receipt or arrival during the time interval 404(1) of whichever pair of possible error codes are represented by whichever two nodes that such directed edge couples together. For instance, suppose that a particular directed edge points away from a node 402(i) and toward a node 402(j), for any suitable positive integers i, j≤p. In such case, this can mean that: whatever possible error code is represented by the node 402(i) is or was produced or generated by the medical imaging scanner 104 during the time interval 404(1) before whatever possible error code represented by the node 402(j) is or was produced or generated by the medical imaging scanner 104 during the time interval 404(1); and no intervening error code was produced after that represented by the node 402(i) but before that represented by the node 402(j).

Note that this can cause the set of directed edges 302(1)(A) to form or trace a contiguous and branchless path through the plurality of nodes 402. Such contiguous and branchless path can be referred to as an order-of-receipt path 302(1)(B), since such path can be considered as conveying the chronological or temporal order in which possible error codes are or were actually produced by the medical imaging scanner 104 during the time interval 404(1).

Furthermore, note that, in situations where multiple error codes are or were produced simultaneously during the time interval 404(1), any one of such multiple error codes can be considered or deemed as predating any other one of such multiple error codes with no intervening error codes in between. For instance, suppose that an error code W is produced during the time interval 404(1), that error codes X and Y are produced simultaneously during the time interval 404(1) after W, and that an error code Z is produced during the time interval 404(1) after X and Y. In such case, the order-of-receipt knowledge graph 302(1) can either: have a first directed edge pointing from W to X, a second directed edge pointing from X to Y, and a third directed edge pointing from Y to Z; or have a first directed edge pointing from W to Y, a second directed edge pointing from Y to X, and a third directed edge pointing from X to Z.

Further still, note that it can be the case that not every possible error code is produced by the medical imaging scanner 104 during the time interval 404(1). In such situations, one or more of the plurality of nodes 402 can be coupled to no edges at all in the order-of-receipt knowledge graph 302(1). In some instances, any nodes of the order-of-receipt knowledge graph 302(1) that are coupled to no edges at all can (but need not) be omitted from the order-of-receipt knowledge graph 302(1).

Conversely, note that it can be the case that some error codes are produced more than once by the medical imaging scanner 104 during the time interval 404(1). For instance, suppose that the error code X is produced during the time interval 404(1), the error code Y is produced during the time interval 404(1) after X, the error code Z is produced during the time interval 404(1) after Y, and the error code X is produced again during the time interval 404(1) after the error code Z. In such situation, the order-of-receipt knowledge graph 302(1) can have a first directed edge pointing away from X toward Y, a second directed edge pointing away from Y toward Z, and a third directed edge pointing away from Z toward X. In other words, it is possible for the order-of-receipt knowledge graph 302(1) to exhibit cycles (e.g., it is possible for the order-of-receipt path 302(1)(B) to have one or more loops along its length).

In any case, the order-of-receipt knowledge graph 302(1) can convey or otherwise represent the chronological or temporal order in which error codes are or were produced by the medical imaging scanner 104 during the time interval 404(1).

As another non-limiting example, the graph component 114 can create an order-of-receipt knowledge graph 302(m) based on the time interval 404(m). Just as above, the order-of-receipt knowledge graph 302(m) can be a digraph that is made up of nodes and directed edges.

More specifically, the order-of-receipt knowledge graph 302(m) can contain the plurality of nodes 402 and can contain a set of directed edges 302(m)(A), which can include any suitable number of directed edges. Just as above, each one of the set of directed edges 302(m) (A) can point or lead away from one respective node in the plurality of nodes 402 and toward another respective node in the plurality of nodes 402, so as to represent a chronological order of receipt or arrival during the time interval 404(m) of whichever pair of possible error codes are represented by those two respective nodes. Thus, just as above, the set of directed edges 302(m) (A) can form or trace a contiguous and branchless path through the plurality of nodes 402. Such contiguous and branchless path can be referred to as an order-of-receipt path 302(m)(B), since such path can be considered as conveying the chronological or temporal order in which possible error codes are or were actually produced by the medical imaging scanner 104 during the time interval 404(m).

Again, note that, in situations where multiple error codes are or were produced simultaneously during the time interval 404(m), any one of such multiple error codes can be considered or deemed as predating any other one of such multiple error codes with no intervening error codes in between.

Additionally, note that it can be the case that not every possible error code is produced by the medical imaging scanner 104 during the time interval 404(m). Any nodes of the order-of-receipt knowledge graph 302(m) that are coupled to no edges at all can (but need not) be omitted from the order-of-receipt knowledge graph 302(m).

Moreover, note that it can be the case that some error codes are produced more than once by the medical imaging scanner 104 during the time interval 404(m). So, it is possible for the order-of-receipt knowledge graph 302(m) to exhibit cycles (e.g., it is possible for the order-of-receipt path 302(m)(B) to have one or more loops along its length).

In any case, the order-of-receipt knowledge graph 302(m) can convey or otherwise represent the chronological or temporal order in which error codes are or were produced by the medical imaging scanner 104 during the time interval 404(m).

In various embodiments, the order-of-receipt knowledge graph 302(1) to the order-of-receipt knowledge graph 302(m) can collectively be considered as forming the order-of-receipt knowledge graph sequence 302.

In various aspects, the order-of-receipt knowledge graph sequence 302 can be considered as better revealing, baring, or uncovering the temporality or chronology of whatever error codes are or were produced by the medical imaging scanner 104 during the retrospective time span 304, as compared to a mere concatenation or list of those error codes.

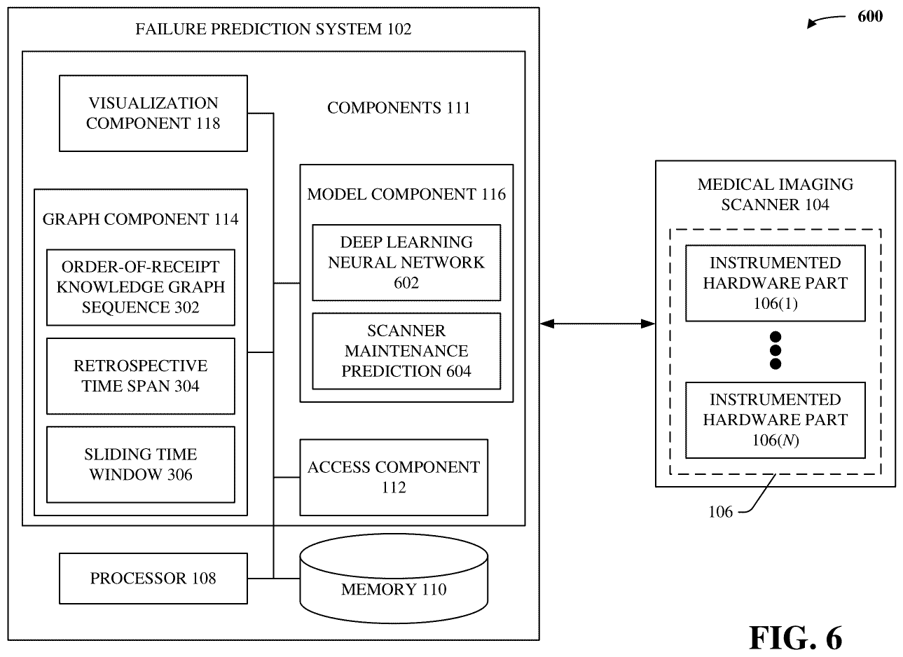

FIG. 6 illustrates a block diagram of an example, non-limiting system 600 including a deep learning neural network and a scanner maintenance prediction that can facilitate failure prediction via graph representation of error codes in accordance with one or more embodiments described herein. As shown, the system 600 can include the same components as the system 300, and can further include a deep learning neural network 602 or a scanner maintenance prediction 604.

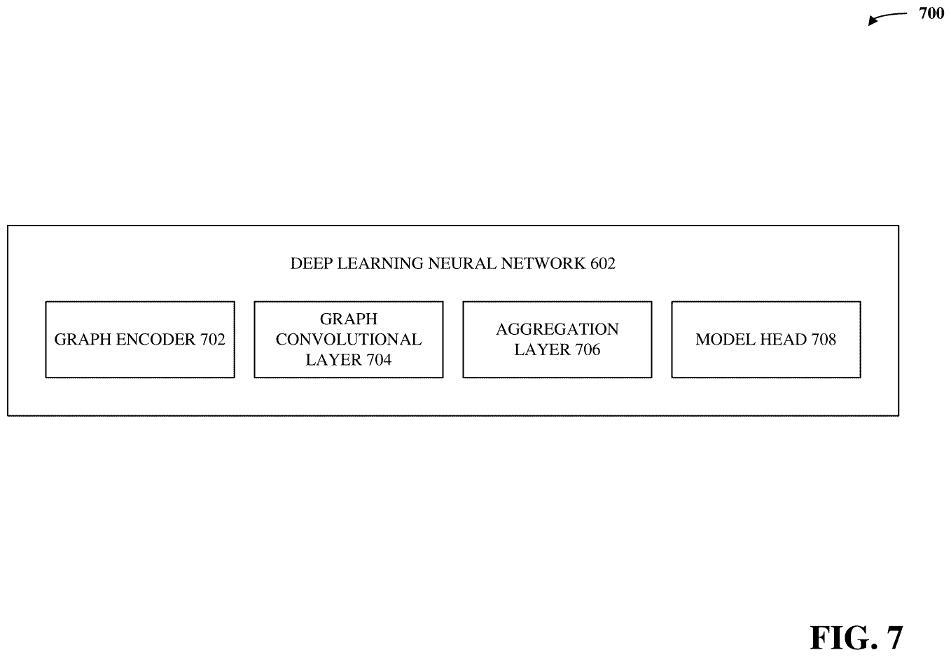

In various embodiments, the model component 116 can electronically store, maintain, control, or otherwise access a deep learning neural network 602. The model component 116 can electronically generate a scanner maintenance prediction 604, based on executing the deep learning neural network 602 on the order-of-receipt knowledge graph sequence 302. Non-limiting aspects are described with respect to FIGS. 7-11.

FIGS. 7-11 illustrate example, non-limiting block diagrams 700, 800, 900, 1000, and 1100 showing how the deep learning neural network 602 can generate the scanner maintenance prediction 604 based on the order-of-receipt knowledge graph sequence 302 in accordance with one or more embodiments described herein.

First, consider FIG. 7. In various embodiments, the deep learning neural network 602 can include a graph encoder 702, a graph convolutional layer 704 that is downstream of the graph encoder 702, an aggregation layer 706 that is downstream of the graph convolutional layer 704, or a model head 708 that is downstream of the aggregation layer 706. In various aspects, execution of the deep learning neural network 602 on the order-of-receipt knowledge graph sequence 302 can involve feeding the order-of-receipt knowledge graph sequence 302 as input to the deep learning neural network 602, such that the order-of-receipt knowledge graph sequence 302 completes a forward pass through the graph encoder 702, through the graph convolutional layer 704, through the aggregation layer 706, and through the model head 708.

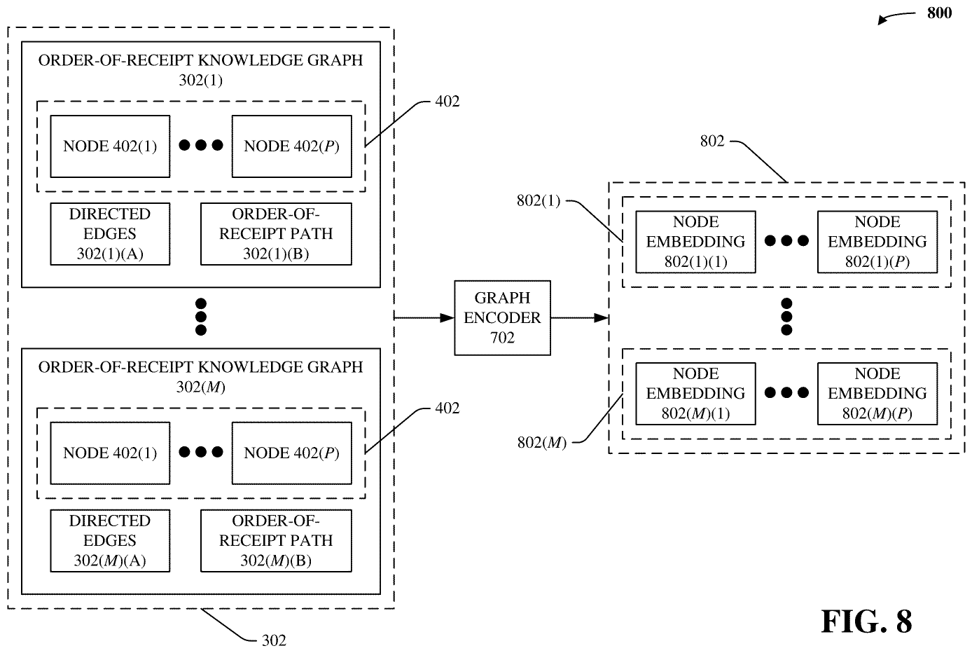

Consider FIG. 8. In various embodiments, the graph encoder 702 can exhibit any suitable deep learning internal architecture. Indeed, in various cases, the graph encoder 702 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be message passing layers, whose learnable or trainable parameters can be node transformation weights, edge transformation weights, or projection weights. As another example, any of such input layer, one or more hidden layers, or output layer can be convolutional layers, whose learnable or trainable parameters can be convolutional kernels. As yet another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. As even another example, any of such input layer, one or more hidden layers, or output layer can be LSTM layers, whose learnable or trainable parameters can be input-state weight matrices or hidden-state weight matrices. As yet another example, any of such input layer, one or more hidden layers, or output layer can be transformer layers, whose learnable or trainable parameters can be single-head or multi-head attention blocks or other weight matrices. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

Regardless of the specific internal architecture (e.g., the specific numbers, types, or organizations of layers) that is implemented within the graph encoder 702, the graph encoder 702 can be configured to receive digraphs and to produce embeddings for the individual nodes of those inputted digraphs. An embedding produced by the graph encoder 702 in response to an inputted digraph can be considered as any suitable mathematical quantity (e.g., scalar, vector, matrix, tensor, or any suitable combination thereof) that numerically represents at least some substantive or semantic aspect of a respective node within that inputted digraph. In other words, the embedding can be considered as a latent vector representation of that respective node. In any case, nodes that are substantively or semantically similar to each other (e.g., that have similar textual payload or numbers or types of edges) can have commensurately similar or close embeddings, whereas nodes that are substantively or semantically dissimilar to each other (e.g., that have dissimilar textual payloads or numbers or types of edges) can have commensurately dissimilar or far-apart embeddings.

In any case, the graph encoder 702 can receive as input the order-of-receipt knowledge graph sequence 302 and can produce as output a sequence of pluralities of node embeddings 802 respectively corresponding to the order-of-receipt knowledge graph sequence 302.

As a non-limiting example, the model component 116 can feed or route the order-of-receipt knowledge graph 302(1) to the input layer of the graph encoder 702. The order-of-receipt knowledge graph 302(1) can complete a forward pass through the one or more hidden layers of the graph encoder 702. The output layer of the graph encoder 702 can compute or otherwise calculate a plurality of node embeddings 802(1) based on whatever activation maps or feature maps are provided by the one or more hidden layers of the graph encoder 702 during such forward pass. In various instances, the plurality of node embeddings 802(1) can have a total cardinality of p, such that each of the plurality of node embeddings 802(1) can be considered as a latent vector representation that the graph encoder 702 believes or infers corresponds to a respective node within the order-of-receipt knowledge graph 302(1). In particular, a node embedding 802(1)(1) can be one or more scalars, one or more vectors, one or more matrices, one or more tensors, or any suitable combination thereof that substantively represents the node 402(1) within the order-of-receipt knowledge graph 302(1). Likewise, a node embedding 802(1)(p) can be one or more scalars, one or more vectors, one or more matrices, one or more tensors, or any suitable combination thereof that substantively represents the node 402(p) within the order-of-receipt knowledge graph 302(1).

As another non-limiting example, the model component 116 can feed or route the order-of-receipt knowledge graph 302(m) to the input layer of the graph encoder 702. The order-of-receipt knowledge graph 302(m) can complete a forward pass through the one or more hidden layers of the graph encoder 702. The output layer of the graph encoder 702 can compute or otherwise calculate a plurality of node embeddings 802(m) based on whatever activation maps or feature maps are provided by the one or more hidden layers of the graph encoder 702 during such forward pass. Just as above, the plurality of node embeddings 802(p) can have a total cardinality of p, such that each of the plurality of node embeddings 802(m) can be considered as a latent vector representation that the graph encoder 702 believes or infers corresponds to a respective node within the order-of-receipt knowledge graph 302(m). In particular, a node embedding 802(m)(1) can be one or more scalars, one or more vectors, one or more matrices, one or more tensors, or any suitable combination thereof that substantively represents the node 402(1) within the order-of-receipt knowledge graph 302(m). Likewise, a node embedding 802(m)(p) can be one or more scalars, one or more vectors, one or more matrices, one or more tensors, or any suitable combination thereof that substantively represents the node 402(p) within the order-of-receipt knowledge graph 302(m).

Note that the plurality of node embeddings 802(1) to the plurality of node embeddings 802(m) can collectively be considered as the sequence of pluralities of node embeddings 802.

Furthermore, note that all of the node embeddings in the sequence of pluralities of node embeddings 802 can have the same size, format, or dimensionality as each other.

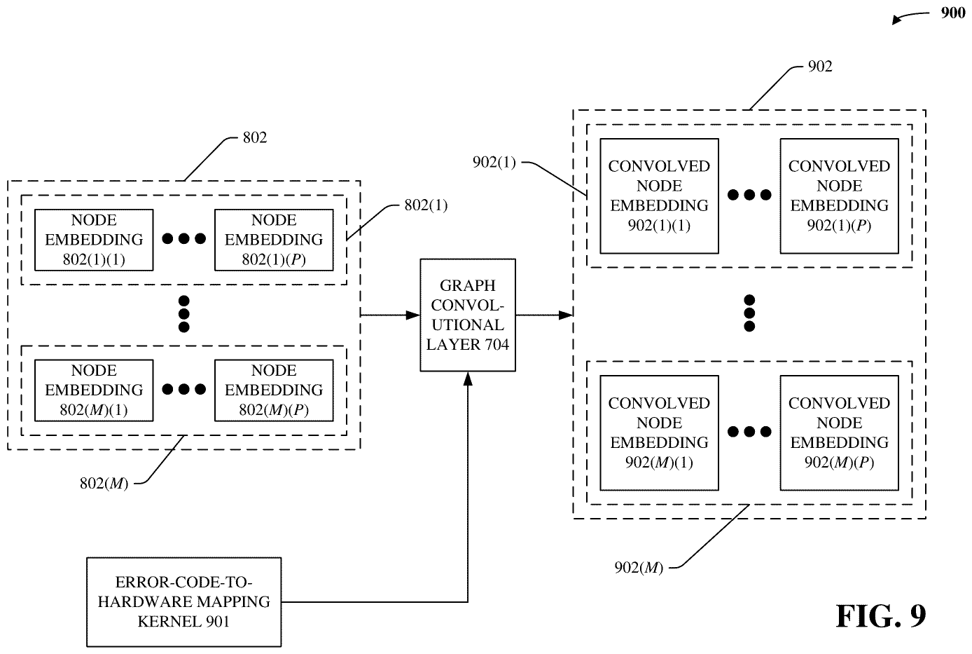

Next, consider FIG. 9. In various embodiments, the graph convolutional layer 704 can be any suitable type of layer that can perform one or more graph convolution operations. Recall that a graph convolution operation can take as input two arguments: a graph; and a convolution kernel, which can be considered as an array indicating, designating, or otherwise representing which nodes of the graph are to be convolved with each other. In various aspects, the convolution kernel utilized by the graph convolutional layer 704 can be an error-code-to-hardware mapping kernel 901, which can be a fixed or non-trainable adjacency matrix or array indicating which error codes (and thus which nodes) correspond to which of the plurality of instrumented hardware parts 106. In other words, the error-code-to-hardware mapping kernel 901 can be considered as conveying, for each particular node of the plurality of nodes 402, which one of the plurality of instrumented hardware parts 106 generated or produced the error code represented by that particular node.

In various instances, the graph convolutional layer 704 can convolve each of the sequence of pluralities of node embeddings 802 with the error-code-to-hardware mapping kernel 901, thereby producing a sequence of pluralities of convolved node embeddings 902.

As a non-limiting example, the model component 116 can feed or route the plurality of node embeddings 802(1) to the graph convolutional layer 704. The graph convolutional layer 704 can, thus, convolve the plurality of node embeddings 802(1) using the error-code-to-hardware mapping kernel 901. In other words, for each given node embedding in the plurality of node embeddings 802(1), that given node embedding can be convolved with whichever others of the plurality of node embeddings 802(1) that correspond to (e.g., that represent error codes produced by) the same instrumented hardware part as the given node embedding (e.g., whichever ones of the plurality of node embeddings 802(1) that correspond to an X-ray tube chiller can all be convolved with each other; whichever ones of the plurality of node embeddings 802(1) that correspond to a gantry control PCB can all be convolved with each other). In any case, the result of such convolution can be referred to as a plurality of convolved node embeddings 902(1). For instance, a convolved node embedding 902(1)(1) can be the convolved version of the node embedding 802(1)(1). Likewise, a convolved node embedding 902(1)(p) can be the convolved version of the node embedding 802(1)(p). In any case, the plurality of node embeddings 802(1) can be considered as latent vector representations that collectively capture the temporality or chronology of whatever error codes are or were produced during the time interval 404(1). In contrast, the plurality of convolved node embeddings 902(1) can be considered as latent vector representations that collectively capture both the temporality or chronology of those error codes as well as the physical or spatial interrelationships between those error codes. That is, the plurality of node embeddings 802(1) can be considered as occupying a temporal domain, whereas the plurality of convolved node embeddings 902(1) can instead be considered as occupying a spatio-temporal domain.

As another non-limiting example, the model component 116 can feed or route the plurality of node embeddings 802(m) to the graph convolutional layer 704. The graph convolutional layer 704 can, thus, convolve the plurality of node embeddings 802(m) using the error-code-to-hardware mapping kernel 901. In other words, for each given node embedding in the plurality of node embeddings 802(m), that given node embedding can be convolved with whichever others of the plurality of node embeddings 802(m) that correspond to (e.g., that represent error codes produced by) the same instrumented hardware part as the given node embedding. As above, the result of such convolution can be referred to as a plurality of convolved node embeddings 902(m). For instance, a convolved node embedding 902(m)(1) can be the convolved version of the node embedding 802(m)(1). Similarly, a convolved node embedding 902(m)(p) can be the convolved version of the node embedding 802(m)(p). Also as above, the plurality of node embeddings 802(m) can be considered as latent vector representations that collectively capture the temporality or chronology of whatever error codes are or were produced during the time interval 404(m) (e.g., as occupying a temporal domain), whereas the plurality of convolved node embeddings 902(m) can be considered as latent vector representations that collectively capture both the temporality or chronology of those error codes as well as the physical or spatial interrelationships between those error codes (e.g., as occupying a spatio-temporal domain).

As above, note that the plurality of convolved node embeddings 902(1) to the plurality of convolved node embeddings 902(m) can be collectively considered as the sequence of pluralities of convolved node embeddings 902.

Also as above, note that each convolved node embedding in the sequence of pluralities of convolved node embeddings 902 can have the same size, format, or dimensionality as each other.

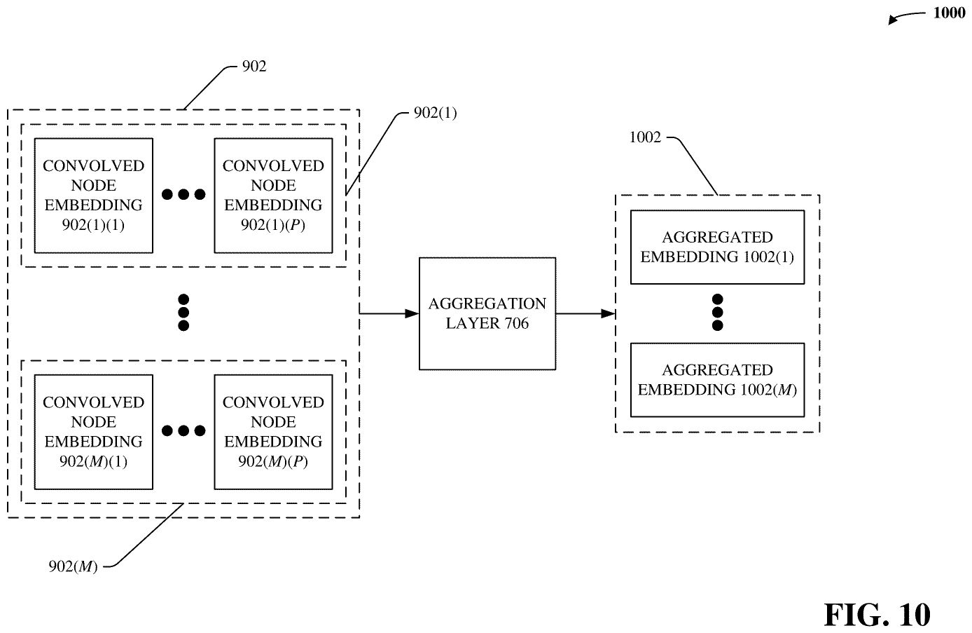

Now, consider FIG. 10. In various embodiments, the aggregation layer 706 can be any suitable type of layer that can perform an aggregation operation. Non-limiting examples of such an aggregation operation can include: a mean or average computation; a standard deviation computation; or a summation computation. In any case, the aggregation layer 706 can aggregate each of the sequence of pluralities of convolved node embeddings 902, thereby producing a sequence of aggregated embeddings 1002.

As a non-limiting example, the model component 116 can feed or route the plurality of convolved node embeddings 902(1) to the aggregation layer 706. The aggregation layer 706 can, thus, aggregate the plurality of convolved node embeddings 902(1) together, thereby yielding an aggregated embedding 1002(1). For instance, the aggregated embedding 1002(1) can be equal to the mean or average of the plurality of convolved node embeddings 902(1). In another instance, the aggregated embedding 1002(1) can be equal to the standard deviation of the plurality of convolved node embeddings 902(1). In yet another instance, the aggregated embedding 1002(1) can be equal to the sum of the plurality of convolved node embeddings 902(1). In any case, the aggregated embedding 1002(1) can be considered as a spatio-temporal latent vector representation of the entirety of the order-of-receipt knowledge graph 302(1), rather than a spatio-temporal latent vector representation of just one node thereof.

As another non-limiting example, the model component 116 can feed or route the plurality of convolved node embeddings 902(m) to the aggregation layer 706. The aggregation layer 706 can, accordingly, aggregate the plurality of convolved node embeddings 902(m) together, thereby yielding an aggregated embedding 1002(m). As above, the aggregated embedding 1002(m) can be equal to the mean or average of, the standard deviation of, or the summation of the plurality of convolved node embeddings 902(m). Also as above, the aggregated embedding 1002(m) can be considered as a spatio-temporal latent vector representation of the entirety of the order-of-receipt knowledge graph 302(m).

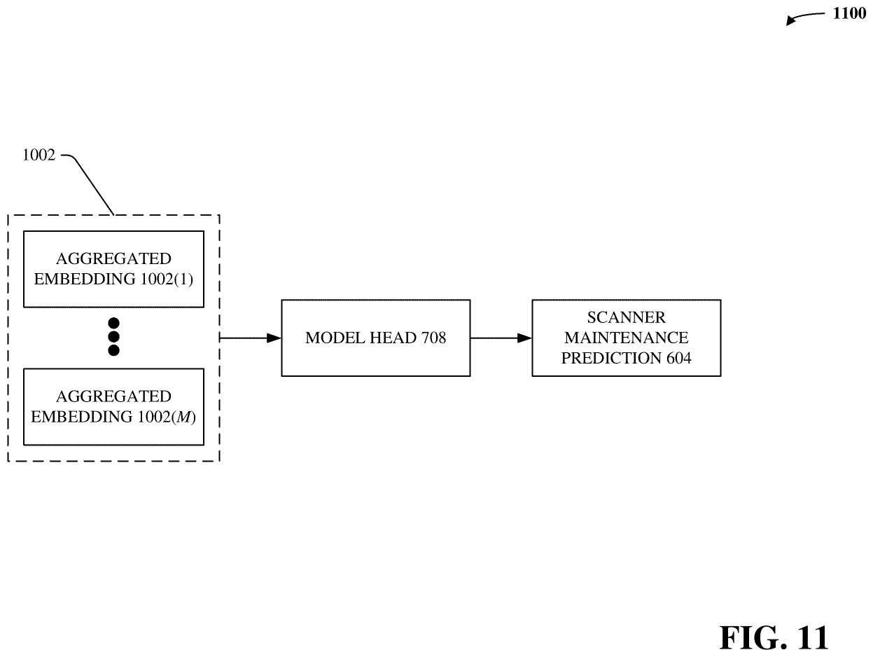

Consider FIG. 11. In various embodiments, the model head 708 can exhibit any suitable deep learning internal architecture. Indeed, in various cases, the model head 708 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. As even another example, any of such input layer, one or more hidden layers, or output layer can be LSTM layers, whose learnable or trainable parameters can be input-state weight matrices or hidden-state weight matrices. As yet another example, any of such input layer, one or more hidden layers, or output layer can be transformer layers, whose learnable or trainable parameters can be single-head or multi-head attention blocks or other weight matrices. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

Regardless of the specific internal architecture (e.g., the specific numbers, types, or organizations of layers) that is implemented within the model head 708, the model head 708 can be configured to receive sequences of aggregated embeddings and to produce maintenance predictions for whatever medical imaging scanners are represented by those inputted sequences of aggregated embeddings. Accordingly, the model head 708 can receive as input the sequence of aggregated embeddings 1002 and can produce as output the scanner maintenance prediction 604. More specifically, the model component 116 can concatenate the sequence of aggregated embeddings 1002 together and can feed or route that concatenation to the input layer of the model head 708. That concatenation can complete a forward pass through the one or more hidden layers of the model head 708. The output layer of the model head 708 can compute or otherwise calculate the scanner maintenance prediction 604 based on whatever activation maps or feature maps are provided by the one or more hidden layers of the model head 708 during such forward pass.

In any case, the scanner maintenance prediction 604 can be any suitable electronic data (e.g., one or more scalars, one or more vectors, one or more matrices, one or more tensors, one or more character strings, or any suitable combination thereof) that represents, indicates, or otherwise conveys some inference regarding an upcoming or impending operational failure of the medical imaging scanner 104. As a non-limiting example, the scanner maintenance prediction 604 can be a classification label, segmentation mask, or regression output that specifies: which particular ones of the plurality of instrumented hardware parts 106 are likely to experience which specific operational failures in the near future; estimated remaining useful lives of those particular instrumented hardware parts (e.g., an inference as to when those particular instrumented hardware parts will experience or suffer those specific operational failures); which particular servicing or maintenance tasks are recommended to be performed on those particular instrumented hardware parts so as to avoid or prevent those specific operational failures; or recommended future times at which those particular servicing or maintenance tasks should be performed so as to avoid or prevent those specific operational failures.

Note that the graph encoder 702, the graph convolutional layer 704, and the aggregation layer 706 can collectively be considered as responsible for formatting or extracting latent features from the order-of-receipt knowledge graph sequence 302. In contrast, note that the model head 708 can instead be considered as responsible for recognizing or detecting within those formatted or extracted latent features whatever subtle signatures signify upcoming or impending operational failures of the medical imaging scanner 104. As explained at length above, an accuracy of the scanner maintenance prediction 604 can be heightened or boosted due to the deep learning neural network 602 being configured to operate on the order-of-receipt knowledge graph sequence 302 (as opposed to a situation in which the deep learning neural network 602 were instead configured to operate on a mere concatenation or list of whatever error codes are or were produced during the retrospective time span 304).

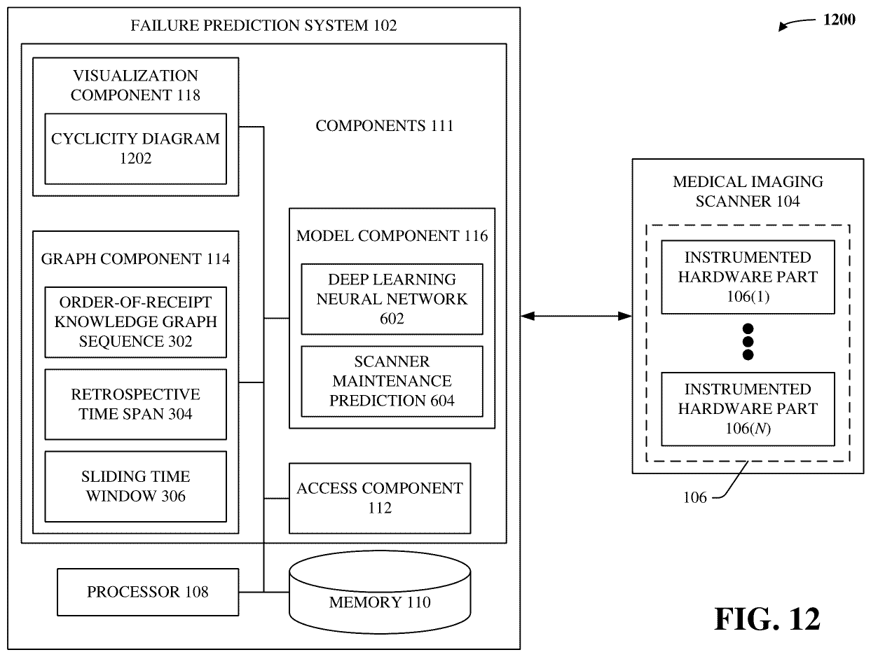

FIG. 12 illustrates a block diagram of an example, non-limiting system 1200 including a cyclicity diagram that can facilitate failure prediction via graph representation of error codes in accordance with one or more embodiments described herein. As shown, the system 1200 can include the same components as the system 600, and can further include a cyclicity diagram 1202.

In various embodiments, the visualization component 118 can initiate or otherwise perform any suitable electronic actions based on the scanner maintenance prediction 604. As a non-limiting example, the visualization component 118 can transmit the scanner maintenance prediction 604 to any suitable computing device. As another non-limiting example, the visualization component 118 can populate an electronic calendar (e.g., of a service technician) with details specified in the scanner maintenance prediction 604. As yet another non-limiting example, the visualization component 118 can render on any suitable computer screen or electronic display the cyclicity diagram 1202. In various aspects, the cyclicity diagram 1202 can be considered as a visual representation of at least one graph in the order-of-receipt knowledge graph sequence 302, where such visual representation can be considered as showing an optical signature or pattern that signifies whatever upcoming or impending operational failures are specified in the scanner maintenance prediction 604. More specifically, for any given order-of-receipt knowledge graph in the order-of-receipt knowledge graph sequence 302, the cyclicity diagram 1202 can include: a plurality of node symbols arranged in a circle or ring, each node symbol representing a respective node of the given order-of-receipt knowledge graph; and a plurality of one-sided arrows located in between respective pairs of node symbols, each one-sided arrow representing a respective directed edge of the given order-of-receipt knowledge graph. With this construction, the cyclicity diagram 1202 can be considered as visually showing the contiguous and branchless path that is traced in the given order-of-receipt knowledge graph. This can be beneficial from an interpretability perspective, because it can be possible for differently shaped order-of-receipt paths to signify different upcoming or impending operational failures (e.g., one particular type of failure might be signified or preceded by order-of-receipt knowledge graphs whose cyclicity diagrams show repetitive loops between error codes produced by X-ray tubes and those produced by X-ray tube control PCBs; whereas a different type of failure might be signified or preceded by order-of-receipt knowledge graphs whose cyclicity diagrams show repetitive loops between error codes produced by X-ray tubes and those produced by X-ray tube chillers). In other words, the cyclicity diagram 1202 can be considered as helping a user or technician associated with the medical imaging scanner 104 to understand why the deep learning neural network 602 produced the scanner maintenance prediction 604.

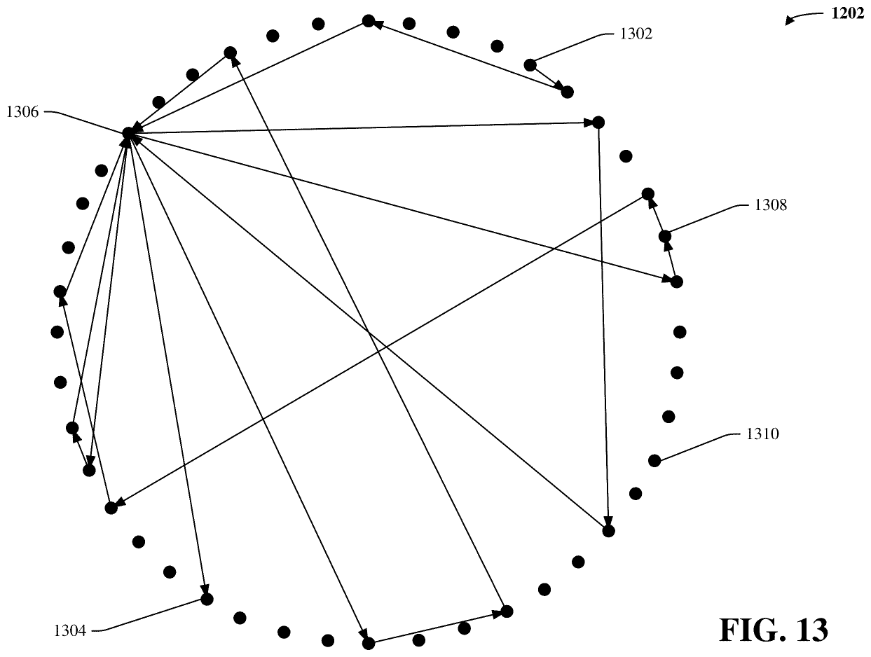

FIG. 13 illustrates an example, non-limiting illustration of the cyclicity diagram 1202 in accordance with one or more embodiments described herein. Each black dot in the non-limiting example of FIG. 13 represents a respective node of the given order-of-receipt knowledge graph. Note that the black dots are arranged circularly or in a ring-like layout. Additionally, each one-sided arrow in the non-limiting example of FIG. 13 represents a respective directed edge of the given order-of-receipt knowledge graph. Note how the arrows form a contiguous and branchless path through the node symbols. Such path represents the temporal or chronological order in which specific error codes are or were produced during whatever time interval corresponds to the given order-of-receipt knowledge graph. In this non-limiting example, such path begins at a node 1302 (e.g., meaning that the error code represented by the node 1302 was the first one produced in that time interval) and ends at a node 1304 (e.g., meaning that the error code represented by the node 1304 was the last one produced in that time interval). As shown, some nodes of the given order-of-receipt knowledge graph have no edges, such as a node 1310. Those nodes can be considered as representing error codes that were not produced during that time interval. As also shown, some nodes of the given order-of-receipt knowledge graph have non-zero but few edges, such as a node 1308. Those nodes can be considered as representing error codes that were produced only a small number of times during that time interval. Additionally, some nodes of the given order-of-receipt knowledge graph have very many edges, such as a node 1306. Those nodes can be considered as representing error codes that were produced many times during that time interval. In some instances, the node symbols (e.g., black dots) can be arranged around the circular or ring-like layout so as to be clustered or grouped according to instrumented hardware part (e.g., such that the error codes producible by one instrumented hardware part are represented only by nodes in an upper left sector of the circular or ring-like layout; such that the error codes producible by another instrumented hardware part are represented only by nodes in a lower right sector of the circular or ring-like layout). So, areas of the cyclicity diagram 1202 that are more densely filled with arrows can be considered as signifying instrumented hardware parts that are or were more vocal in terms of error code generation during the associated time interval, whereas areas of the cyclicity diagram 1202 that are less densely filled with arrows can be considered as signifying instrumented hardware parts that are or were less vocal in terms of error code generation during that associated time interval.

In some situations, the visualization component 118 can generate or render a respective cyclicity diagram for each order-of-receipt knowledge graph in the order-of-receipt knowledge graph sequence 302, thereby yielding a sequence of cyclicity diagrams. In some of such cases, the visualization component 118 can play or animate the sequence of cyclicity diagrams by superimposing them one-by-one on top of each other or otherwise showing one of them at a time in quick succession. Such playing or animation can help to visually show to the user or technician how the temporality or chronology of the error codes produced during the retrospective time span 304 changes from interval to interval.

In order for the scanner maintenance prediction 604 to be correct, accurate, or otherwise reliable, the deep learning neural network 602 can first undergo training.

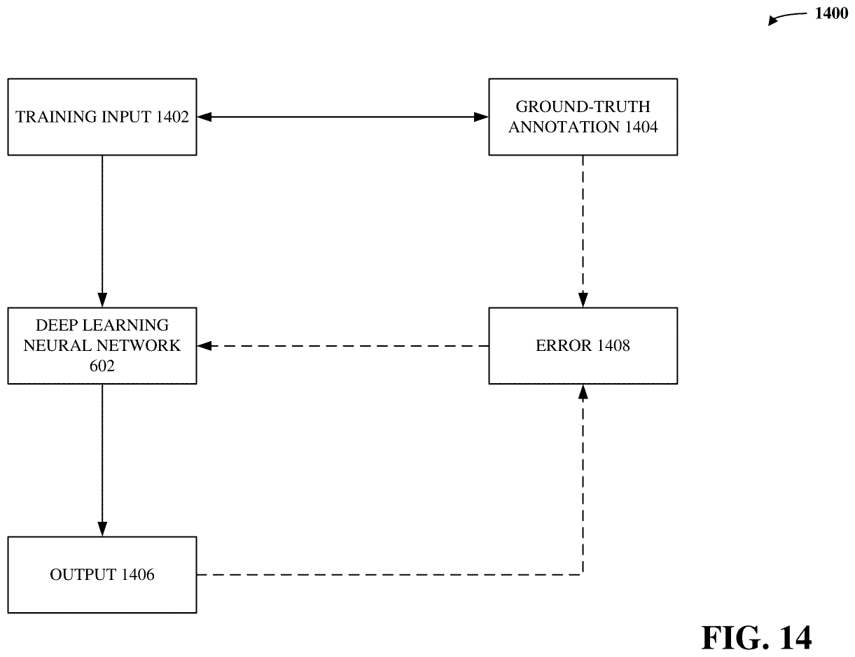

FIG. 14 illustrates an example, non-limiting block diagram 1400 showing how the deep learning neural network 602 can be trained in accordance with one or more embodiments described herein.

In various aspects, prior to beginning training, the trainable internal parameters (e.g., convolutional kernels, weight matrices, bias values, projection weights) of the deep learning neural network 602 can be initialized in any suitable fashion (e.g., via random initialization) by the failure prediction system 102.

In various embodiments, there can be a training input 1402 and a ground-truth annotation 1404. The training input 1402 can have the same format, size, or dimensionality as the order-of-receipt knowledge graph sequence 302. In contrast, the ground-truth annotation 1404 can have the same format, size, or dimensionality as the scanner maintenance prediction 604. In other words, the training input 1402 can be any suitable historical sequence of knowledge graphs constructed as described herein for the medical imaging scanner 104, and the ground-truth annotation 1404 can be whatever correct or accurate maintenance prediction is known or deemed to correspond to that historical sequence (e.g., can indicate whatever operational failure or maintenance recommendation historically transpired after whatever retrospective time span that is represented by that historical sequence).

In any case, the failure prediction system 102 can cause the deep learning neural network 602 to be executed on the training input 1402, thereby causing the deep learning neural network 602 to produce an output 1406. More specifically, the training input 1402 can complete a forward pass through the graph encoder 702, thereby yielding a sequence of pluralities of node embeddings. Next, the graph convolutional layer 704 can convolve those node embeddings with the error-code-to-hardware mapping kernel 901, thereby yielding a sequence of pluralities of convolved node embeddings. Then, the aggregation layer 706 can aggregate those convolved node embeddings together, thereby yielding a sequence of aggregated embeddings. Finally, the sequence of aggregated embeddings can be concatenated together and complete a forward pass through the model head 708, thereby yielding the output 1406.

Note that the format, size, or dimensionality of the output 1406 can be dictated by the number, arrangement, sizes, or other characteristics of the neurons, convolutional kernels, attention blocks, or other internal parameters of the output layer (or of any other layers) of the deep learning neural network 602. Accordingly, the output 1406 can be forced to have any desired format, size, or dimensionality (e.g., forced to have the same format, size, or dimensionality as the ground-truth annotation 1404), by adding, removing, or otherwise adjusting characteristics of the output layer (or of any other layers) of the deep learning neural network 602. So, the output 1406 can be considered as the inferred scanner maintenance prediction that the deep learning neural network 602 has created based on the training input 1402. In contrast, the ground-truth annotation 1404 can be considered as whatever correct or accurate scanner maintenance prediction that is known or deemed to correspond to the training input 1402. Note that, if the deep learning neural network 602 has so far undergone no or little training, then the output 1406 can be highly inaccurate. In other words, the output 1406 can be very different from the ground-truth annotation 1404.

In various aspects, an error 1408 (e.g., mean absolute error, mean squared error, cross-entropy error) between the output 1406 and the ground-truth annotation 1404 can be computed by the failure prediction system 102. The trainable internal parameters of the deep learning neural network 602 can be incrementally updated via backpropagation (e.g., stochastic gradient descent) based on the error 1408.

Such execution-and-update procedure can be repeated for any suitable number input-annotation pairs. This can ultimately cause the trainable internal parameters of the deep learning neural network 602 to become iteratively optimized for accurately predicting maintenance information (e.g., remaining useful life of specified instrumented hardware parts, recommended maintenance tasks to perform on specified instrumented hardware parts) for the medical imaging scanner 104. Any suitable training batch sizes, any suitable error/loss functions, or any suitable training termination criteria can be utilized during such training.

Although the herein disclosure mainly describes the deep learning neural network 602 as being trained in supervised fashion, this is a mere non-limiting example for ease of explanation and illustration. In various embodiments, any other suitable training paradigms can be used to train the deep learning neural network 602, such as unsupervised training, semi-supervised training, reinforcement learning, or transfer learning, any of which may be federated or unfederated.

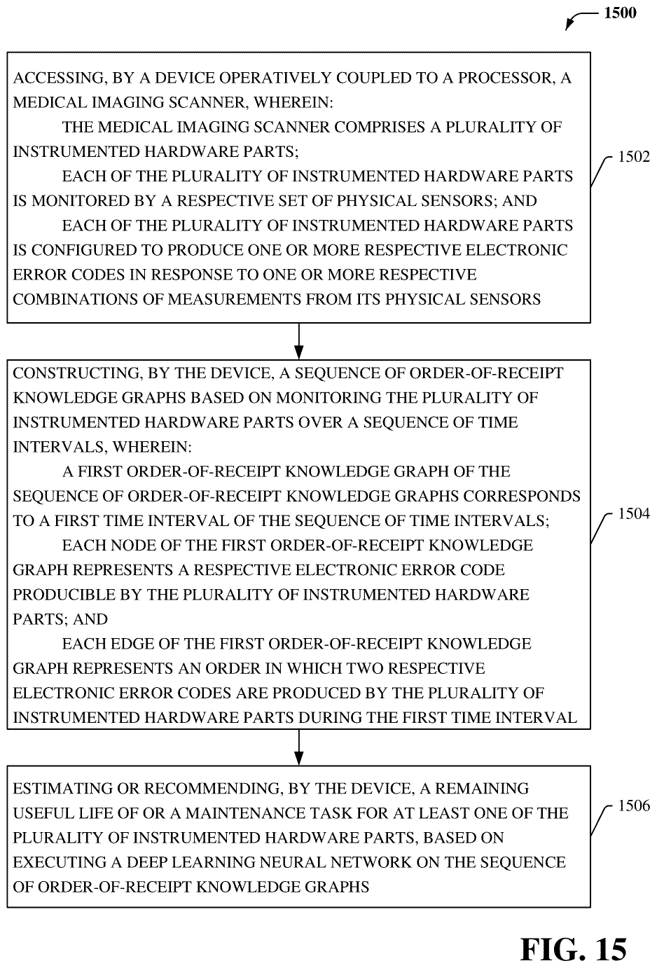

FIG. 15 illustrates a flow diagram of an example, non-limiting computer-implemented method 1500 that can facilitate failure prediction via graph representation of error codes in accordance with one or more embodiments described herein. In various cases, the failure prediction system 102 can facilitate the computer-implemented method 1500.

In various embodiments, act 1502 can include accessing, by a device (e.g., via 112) operatively coupled to a processor (e.g., 108), a medical imaging scanner (e.g., 104). In various aspects, it can be the case that: the medical imaging scanner includes a plurality of instrumented hardware parts (e.g., 106); each of the plurality of instrumented hardware parts is monitored by a respective set of physical sensors; and each of the plurality of instrumented hardware parts is configured to produce one or more respective electronic error codes (e.g., 202) in response to one or more respective combinations of measurements from its physical sensors.

In various instances, act 1504 can include constructing, by the device (e.g., via 114), a sequence of order-of-receipt knowledge graphs (e.g., 302) based on monitoring the plurality of instrumented hardware parts over a sequence of time intervals (e.g., 404). In various cases, it can be the case that: a first order-of-receipt knowledge graph (e.g., 302(m)) of the sequence of order-of-receipt knowledge graphs corresponds to a first time interval (e.g., 404(m)) of the sequence of time intervals; each node (e.g., 402) of the first order-of-receipt knowledge graph represents a respective electronic error code (e.g., 202(m)(1)) producible by the plurality of instrumented hardware parts; and each edge (e.g., 302(m)(A)) of the first order-of-receipt knowledge graph represents an order in which two respective electronic error codes are produced by the plurality of instrumented hardware parts during the first time interval.

In various aspects, act 1506 can include estimating or recommending, by the device (e.g., via 116), a remaining useful life of or a maintenance task (e.g., 604) for at least one of the plurality of instrumented hardware parts, based on executing a deep learning neural network (e.g., 602) on the sequence of order-of-receipt knowledge graphs.

Although not explicitly shown in FIG. 15, the deep learning neural network can include a graph encoder (e.g., 702) that receives as input the sequence of order-of-receipt knowledge graphs and computes a respective embedding (e.g., 802) for each node of each one of the sequence of order-of-receipt knowledge graphs.

Although not explicitly shown in FIG. 15, the deep learning neural network can include a graph convolutional layer (e.g., 704) downstream of the graph encoder. In various cases, for each order-of-receipt knowledge graph in the sequence of order-of-receipt knowledge graphs, the graph convolutional layer can perform a convolution between: node embeddings (e.g., 802) produced by the graph encoder for that order-of-receipt knowledge graph; and a kernel (e.g., 901) indicating to which of the plurality of instrumented hardware parts respective error codes belong.

Although not explicitly shown in FIG. 15, the deep learning neural network can include an aggregation layer (e.g., 706) that is downstream of the graph convolutional layer. In various instances, for each order-of-receipt knowledge graph in the sequence of order-of-receipt knowledge graphs, the aggregation layer can aggregate together convolved node embeddings (e.g., 902) produced by the graph convolutional layer for that order-of-receipt knowledge graph, thereby yielding a sequence of aggregated embeddings (e.g., 1002) respectively corresponding to the sequence of order-of-receipt knowledge graphs. In various aspects, the aggregation layer can aggregate convolved node embeddings via at least one from the group consisting of: a sum computation; an average computation; and a standard deviation computation.

Although not explicitly shown in FIG. 15, the deep learning neural network can include a model head (e.g., 708) that receives the sequence of aggregated embeddings and computes as output an indicator (e.g., 604) indicating the remaining useful life or maintenance task.

Although not explicitly shown in FIG. 15, the computer-implemented method 1500 can include: rendering, by the device (e.g., via 118) and on an electronic display: an indication of the remaining useful life or maintenance task (e.g., rendering 604); or a cyclicity diagram (e.g., 1202) that is formed based on illustrating at least one of the sequence of order-of-receipt knowledge graphs such that its nodes are visually depicted in a circular layout.

Although not explicitly shown in FIG. 15, the sequence of time intervals can be formed by scrubbing in defined temporal increments (e.g., 504) a sliding window (e.g., 306) across a time span (e.g., 304) measured backward from a current time (e.g., 502).

Although various embodiments are described herein as relating to medical imaging scanners, these are mere non-limiting examples for ease of explanation and illustration. Indeed, various embodiments described herein can be readily extrapolated so as to facilitate improved or higher-accuracy failure prediction for any suitable machines or devices that are constructed or made up of instrumented hardware parts, such as vehicles, home appliances, or fabrication equipment. In other words, various embodiments described herein can be not limited to medical imaging scanners.

In various instances, machine learning algorithms or models can be implemented in any suitable way to facilitate any suitable aspects described herein. To facilitate some of the above-described machine learning aspects of various embodiments, consider the following discussion of artificial intelligence (AI). Various embodiments described herein can employ artificial intelligence to facilitate automating one or more features or functionalities. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute) described herein, components described herein can examine the entirety or a subset of the data to which it is granted access and can provide for reasoning about or determine states of the system or environment from a set of observations as captured via events or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events or data.

Such determinations can result in the construction of new events or actions from a set of observed events or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes or systems (e.g., support vector machines, neural networks, expert systems, Bayesian belief networks, fuzzy logic, data fusion engines, and so on) in connection with performing automatic or determined action in connection with the claimed subject matter. Thus, classification schemes or systems can be used to automatically learn and perform a number of functions, actions, or determinations.

A classifier can map an input attribute vector, z=(z₁, z₂, z₃, z₄, zₙ), to a confidence that the input belongs to a class, as by f(z)=confidence(class). Such classification can employ a probabilistic or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, or probabilistic classification models providing different patterns of independence, any of which can be employed. Classification as used herein also is inclusive of statistical regression that is utilized to develop models of priority.

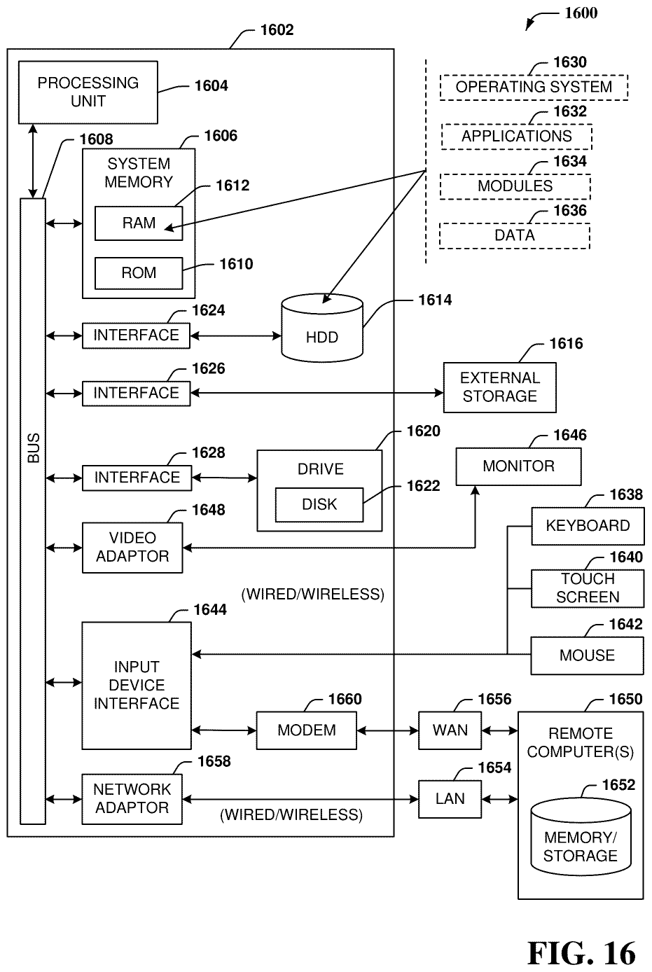

In order to provide additional context for various embodiments described herein, FIG. 16 and the following discussion are intended to provide a brief, general description of a suitable computing environment 1600 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 16, the example environment 1600 for implementing various embodiments of the aspects described herein includes a computer 1602, the computer 1602 including a processing unit 1604, a system memory 1606 and a system bus 1608. The system bus 1608 couples system components including, but not limited to, the system memory 1606 to the processing unit 1604. The processing unit 1604 can be any of various commercially available processors. Dual microprocessors and other multi-processor architectures can also be employed as the processing unit 1604.

The system bus 1608 can be any of several types of bus structure that can further interconnect to a memory bus (with or without a memory controller), a peripheral bus, and a local bus using any of a variety of commercially available bus architectures. The system memory 1606 includes ROM 1610 and RAM 1612. A basic input/output system (BIOS) can be stored in a non-volatile memory such as ROM, erasable programmable read only memory (EPROM), EEPROM, which BIOS contains the basic routines that help to transfer information between elements within the computer 1602, such as during startup. The RAM 1612 can also include a high-speed RAM such as static RAM for caching data.

The computer 1602 further includes an internal hard disk drive (HDD) 1614 (e.g., EIDE, SATA), one or more external storage devices 1616 (e.g., a magnetic floppy disk drive (FDD) 1616, a memory stick or flash drive reader, a memory card reader, etc.) and a drive 1620, e.g., such as a solid state drive, an optical disk drive, which can read or write from a disk 1622, such as a CD-ROM disc, a DVD, a BD, etc. Alternatively, where a solid state drive is involved, disk 1622 would not be included, unless separate. While the internal HDD 1614 is illustrated as located within the computer 1602, the internal HDD 1614 can also be configured for external use in a suitable chassis (not shown). Additionally, while not shown in environment 1600, a solid state drive (SSD) could be used in addition to, or in place of, an HDD 1614. The HDD 1614, external storage device(s) 1616 and drive 1620 can be connected to the system bus 1608 by an HDD interface 1624, an external storage interface 1626 and a drive interface 1628, respectively. The interface 1624 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1602, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1612, including an operating system 1630, one or more application programs 1632, other program modules 1634 and program data 1636. All or portions of the operating system, applications, modules, or data can also be cached in the RAM 1612. The systems and methods described herein can be implemented utilizing various commercially available operating systems or combinations of operating systems.

Computer 1602 can optionally comprise emulation technologies. For example, a hypervisor (not shown) or other intermediary can emulate a hardware environment for operating system 1630, and the emulated hardware can optionally be different from the hardware illustrated in FIG. 16. In such an embodiment, operating system 1630 can comprise one virtual machine (VM) of multiple VMs hosted at computer 1602. Furthermore, operating system 1630 can provide runtime environments, such as the Java runtime environment or the .NET framework, for applications 1632. Runtime environments are consistent execution environments that allow applications 1632 to run on any operating system that includes the runtime environment. Similarly, operating system 1630 can support containers, and applications 1632 can be in the form of containers, which are lightweight, standalone, executable packages of software that include, e.g., code, runtime, system tools, system libraries and settings for an application.

Further, computer 1602 can be enable with a security module, such as a trusted processing module (TPM). For instance with a TPM, boot components hash next in time boot components, and wait for a match of results to secured values, before loading a next boot component. This process can take place at any layer in the code execution stack of computer 1602, e.g., applied at the application execution level or at the operating system (OS) kernel level, thereby enabling security at any level of code execution.

A user can enter commands and information into the computer 1602 through one or more wired/wireless input devices, e.g., a keyboard 1638, a touch screen 1640, and a pointing device, such as a mouse 1642. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1604 through an input device interface 1644 that can be coupled to the system bus 1608, but can be connected by other interfaces, such as a parallel port, an IEEE 1394 serial port, a game port, a USB port, an IR interface, a BLUETOOTH® interface, etc.

A monitor 1646 or other type of display device can be also connected to the system bus 1608 via an interface, such as a video adapter 1648. In addition to the monitor 1646, a computer typically includes other peripheral output devices (not shown), such as speakers, printers, etc.

The computer 1602 can operate in a networked environment using logical connections via wired or wireless communications to one or more remote computers, such as a remote computer(s) 1650. The remote computer(s) 1650 can be a workstation, a server computer, a router, a personal computer, portable computer, microprocessor-based entertainment appliance, a peer device or other common network node, and typically includes many or all of the elements described relative to the computer 1602, although, for purposes of brevity, only a memory/storage device 1652 is illustrated. The logical connections depicted include wired/wireless connectivity to a local area network (LAN) 1654 or larger networks, e.g., a wide area network (WAN) 1656. Such LAN and WAN networking environments are commonplace in offices and companies, and facilitate enterprise-wide computer networks, such as intranets, all of which can connect to a global communications network, e.g., the Internet.

When used in a LAN networking environment, the computer 1602 can be connected to the local network 1654 through a wired or wireless communication network interface or adapter 1658. The adapter 1658 can facilitate wired or wireless communication to the LAN 1654, which can also include a wireless access point (AP) disposed thereon for communicating with the adapter 1658 in a wireless mode.

When used in a WAN networking environment, the computer 1602 can include a modem 1660 or can be connected to a communications server on the WAN 1656 via other means for establishing communications over the WAN 1656, such as by way of the Internet. The modem 1660, which can be internal or external and a wired or wireless device, can be connected to the system bus 1608 via the input device interface 1644. In a networked environment, program modules depicted relative to the computer 1602 or portions thereof, can be stored in the remote memory/storage device 1652. It will be appreciated that the network connections shown are example and other means of establishing a communications link between the computers can be used.

When used in either a LAN or WAN networking environment, the computer 1602 can access cloud storage systems or other network-based storage systems in addition to, or in place of, external storage devices 1616 as described above, such as but not limited to a network virtual machine providing one or more aspects of storage or processing of information. Generally, a connection between the computer 1602 and a cloud storage system can be established over a LAN 1654 or WAN 1656 e.g., by the adapter 1658 or modem 1660, respectively. Upon connecting the computer 1602 to an associated cloud storage system, the external storage interface 1626 can, with the aid of the adapter 1658 or modem 1660, manage storage provided by the cloud storage system as it would other types of external storage. For instance, the external storage interface 1626 can be configured to provide access to cloud storage sources as if those sources were physically connected to the computer 1602.

The computer 1602 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

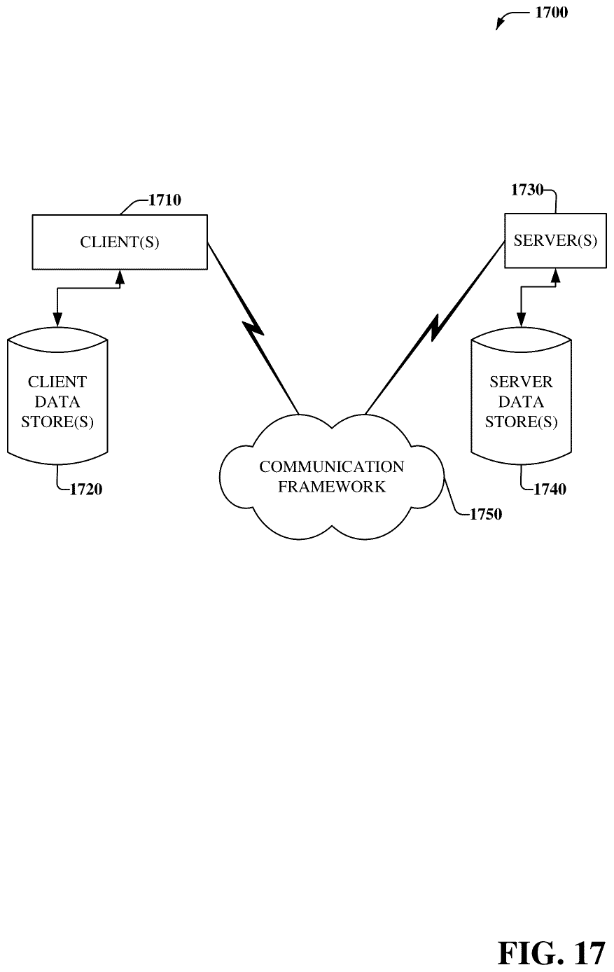

FIG. 17 is a schematic block diagram of a sample computing environment 1700 with which the disclosed subject matter can interact. The sample computing environment 1700 includes one or more client(s) 1710. The client(s) 1710 can be hardware or software (e.g., threads, processes, computing devices). The sample computing environment 1700 also includes one or more server(s) 1730. The server(s) 1730 can also be hardware or software (e.g., threads, processes, computing devices). The servers 1730 can house threads to perform transformations by employing one or more embodiments as described herein, for example. One possible communication between a client 1710 and a server 1730 can be in the form of a data packet adapted to be transmitted between two or more computer processes. The sample computing environment 1700 includes a communication framework 1750 that can be employed to facilitate communications between the client(s) 1710 and the server(s) 1730. The client(s) 1710 are operably connected to one or more client data store(s) 1720 that can be employed to store information local to the client(s) 1710. Similarly, the server(s) 1730 are operably connected to one or more server data store(s) 1740 that can be employed to store information local to the servers 1730.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a solid state drive such as M.2 (including non-volatile memory express (NVMe) or serial advanced technology attachment (SATA)), a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various non-limiting aspects are described in the following clauses.
CLAUSE 1: A system can comprise: a processor that can execute computer-executable components stored in a non-transitory computer-readable memory, wherein the computer-executable components can comprise: an access component that can access a medical imaging scanner, wherein: the medical imaging scanner can comprise a plurality of instrumented hardware parts; each of the plurality of instrumented hardware parts can be monitored by a respective set of physical sensors; and each of the plurality of instrumented hardware parts can be configured to produce one or more respective electronic error codes in response to one or more respective combinations of measurements from its physical sensors; a graph component that can construct a sequence of order-of-receipt knowledge graphs based on monitoring the plurality of instrumented hardware parts over a sequence of time intervals, wherein: a first order-of-receipt knowledge graph of the sequence of order-of-receipt knowledge graphs can correspond to a first time interval of the sequence of time intervals; each node of the first order-of-receipt knowledge graph can represent a respective electronic error code producible by the plurality of instrumented hardware parts; and each edge of the first order-of-receipt knowledge graph can represent an order in which two respective electronic error codes are produced by the plurality of instrumented hardware parts during the first time interval; and a model component that can estimate a remaining useful life of or recommends a maintenance task for at least one of the plurality of instrumented hardware parts, based on executing a deep learning neural network on the sequence of order-of-receipt knowledge graphs.
CLAUSE 2: The system of any preceding clause can be implemented, wherein the deep learning neural network can comprise a graph encoder that can receive as input the sequence of order-of-receipt knowledge graphs and compute a respective embedding for each node of each one of the sequence of order-of-receipt knowledge graphs.
CLAUSE 3: The system of any preceding clause can be implemented, wherein the deep learning neural network can comprise a graph convolutional layer downstream of the graph encoder, and wherein, for each order-of-receipt knowledge graph in the sequence of order-of-receipt knowledge graphs, the graph convolutional layer can perform a convolution between: node embeddings produced by the graph encoder for that order-of-receipt knowledge graph; and a kernel indicating to which of the plurality of instrumented hardware parts respective error codes belong.
CLAUSE 4: The system of any preceding clause can be implemented, wherein the deep learning neural network can comprise an aggregation layer that can be downstream of the graph convolutional layer, and wherein, for each order-of-receipt knowledge graph in the sequence of order-of-receipt knowledge graphs, the aggregation layer can aggregate together convolved node embeddings produced by the graph convolutional layer for that order-of-receipt knowledge graph, thereby yielding a sequence of aggregated embeddings respectively corresponding to the sequence of order-of-receipt knowledge graphs.
CLAUSE 5: The system of any preceding clause can be implemented, wherein the aggregation layer can aggregate convolved node embeddings via at least one from the group consisting of: a sum computation; an average computation; and a standard deviation computation.
CLAUSE 6: The system of any preceding clause can be implemented, wherein the deep learning neural network can comprise a model head that can receive the sequence of aggregated embeddings and compute as output an indicator indicating the remaining useful life or maintenance task.
CLAUSE 7: The system of any preceding clause can be implemented, wherein the medical imaging scanner can comprise at least one from the group consisting of: a computed tomography scanner; an X-ray scanner; an ultrasound scanner; a magnetic resonance imaging scanner; a positron emission tomography scanner; a nuclear medicine scanner; and an image-guided surgery machine.
CLAUSE 8: The system of any preceding clause can be implemented, wherein the computer-executable components can comprise: a visualization component that can render on an electronic display: an indication of the remaining useful life or maintenance task; or a cyclicity diagram that can be formed based on illustrating at least one of the sequence of order-of-receipt knowledge graphs such that its nodes are visually depicted in a circular layout.
CLAUSE 9: The system of any preceding clause can be implemented, wherein the sequence of time intervals can be formed by scrubbing in defined temporal increments a sliding window across a time span measured backward from a current time

In various aspects, any combination or combinations of Clauses 1-9 can be implemented.
CLAUSE 10: A computer-implemented method can comprise: accessing, by a device operatively coupled to a processor, a medical imaging scanner, wherein: the medical imaging scanner can comprise a plurality of instrumented hardware parts; each of the plurality of instrumented hardware parts can be monitored by a respective set of physical sensors; and each of the plurality of instrumented hardware parts can be configured to produce one or more respective electronic error codes in response to one or more respective combinations of measurements from its physical sensors; constructing, by the device, a sequence of order-of-receipt knowledge graphs based on monitoring the plurality of instrumented hardware parts over a sequence of time intervals, wherein: a first order-of-receipt knowledge graph of the sequence of order-of-receipt knowledge graphs can correspond to a first time interval of the sequence of time intervals; each node of the first order-of-receipt knowledge graph can represent a respective electronic error code producible by the plurality of instrumented hardware parts; and each edge of the first order-of-receipt knowledge graph can represent an order in which two respective electronic error codes are produced by the plurality of instrumented hardware parts during the first time interval; and estimating or recommending, by the device, a remaining useful life of or a maintenance task for at least one of the plurality of instrumented hardware parts, based on executing a deep learning neural network on the sequence of order-of-receipt knowledge graphs.
CLAUSE 11: The computer-implemented method of any preceding clause can be implemented, wherein the deep learning neural network can comprise a graph encoder that can receive as input the sequence of order-of-receipt knowledge graphs and compute a respective embedding for each node of each one of the sequence of order-of-receipt knowledge graphs.
CLAUSE 12: The computer-implemented method of any preceding clause can be implemented, wherein the deep learning neural network can comprise a graph convolutional layer downstream of the graph encoder, and wherein, for each order-of-receipt knowledge graph in the sequence of order-of-receipt knowledge graphs, the graph convolutional layer can perform a convolution between: node embeddings produced by the graph encoder for that order-of-receipt knowledge graph; and a kernel indicating to which of the plurality of instrumented hardware parts respective error codes belong.
CLAUSE 13: The computer-implemented method of any preceding claim can be implemented, wherein the deep learning neural network can comprise an aggregation layer that can be downstream of the graph convolutional layer, and wherein, for each order-of-receipt knowledge graph in the sequence of order-of-receipt knowledge graphs, the aggregation layer can aggregate together convolved node embeddings produced by the graph convolutional layer for that order-of-receipt knowledge graph, thereby yielding a sequence of aggregated embeddings respectively corresponding to the sequence of order-of-receipt knowledge graphs.
CLAUSE 14: The computer-implemented method of any preceding clause can be implemented, wherein the aggregation layer can aggregate convolved node embeddings via at least one from the group consisting of: a sum computation; an average computation; and a standard deviation computation.
CLAUSE 15: The computer-implemented method of any preceding clause can be implemented, wherein the deep learning neural network can comprise a model head that can receive the sequence of aggregated embeddings and compute as output an indicator indicating the remaining useful life or maintenance task.
CLAUSE 16: The computer-implemented method of any preceding clause can be implemented, wherein the medical imaging scanner can comprise at least one from the group consisting of: a computed tomography scanner; an X-ray scanner; an ultrasound scanner; a magnetic resonance imaging scanner; a positron emission tomography scanner; a nuclear medicine scanner; and an image-guided surgery machine.
CLAUSE 17: The computer-implemented method of any preceding clause can be implemented, further comprising: rendering, by the device and on an electronic display: an indication of the remaining useful life or maintenance task; or a cyclicity diagram that can be formed based on illustrating at least one of the sequence of order-of-receipt knowledge graphs such that its nodes are visually depicted in a circular layout.
CLAUSE 18: The computer-implemented method of any preceding clause can be implemented, wherein the sequence of time intervals can be formed by scrubbing in defined temporal increments a sliding window across a time span measured backward from a current time.

In various aspects, any combination or combinations of Clauses 10-18 can be implemented.
CLAUSE 19: A computer program product for facilitating failure prediction via graph representation of error codes can be implemented, wherein the computer program product can comprise a non-transitory computer-readable memory having program instructions embodied therewith, and wherein the program instructions can be executable by a processor to cause the processor to: access a machine, wherein: the machine can comprise a plurality of instrumented hardware parts; each of the plurality of instrumented hardware parts can be monitored by a respective set of physical sensors; and each of the plurality of instrumented hardware parts can be configured to produce one or more respective electronic error codes in response to one or more respective combinations of measurements from its physical sensors; construct a sequence of knowledge graphs based on monitoring the plurality of instrumented hardware parts over a sequence of time intervals, wherein: a first knowledge graph of the sequence of knowledge graphs can correspond to a first time interval of the sequence of time intervals; each node of the first knowledge graph can represent a respective electronic error code producible by the plurality of instrumented hardware parts; and each edge of the first knowledge graph can represent an order in which two respective electronic error codes are produced by the plurality of instrumented hardware parts during the first time interval; and estimate or recommend a remaining useful life of or a maintenance task for at least one of the plurality of instrumented hardware parts, based on executing a deep learning neural network on the sequence of knowledge graphs.
CLAUSE 20: The computer program product of any preceding clause can be implemented, wherein the deep learning neural network can convolve each of the sequence of knowledge graphs with a kernel indicating to which of the plurality of instrumented hardware parts respective error codes belong.

In various aspects, any combination or combinations of Clauses 19-20 can be implemented.

In various aspects, any combination or combinations of Clauses 1-20 can be implemented.

What is claimed is:

1. A system, comprising:
a processor that executes computer-executable components stored in a non-transitory computer-readable memory, wherein the computer-executable components comprise:
an access component that accesses a medical imaging scanner, wherein:
the medical imaging scanner comprises a plurality of instrumented hardware parts;
each of the plurality of instrumented hardware parts is monitored by a respective set of physical sensors; and
each of the plurality of instrumented hardware parts is configured to produce one or more respective electronic error codes in response to one or more respective combinations of measurements from its physical sensors;
a graph component that constructs a sequence of order-of-receipt knowledge graphs based on monitoring the plurality of instrumented hardware parts over a sequence of time intervals, wherein:
a first order-of-receipt knowledge graph of the sequence of order-of-receipt knowledge graphs corresponds to a first time interval of the sequence of time intervals;
each node of the first order-of-receipt knowledge graph represents a respective electronic error code producible by the plurality of instrumented hardware parts; and
each edge of the first order-of-receipt knowledge graph represents an order in which two respective electronic error codes are produced by the plurality of instrumented hardware parts during the first time interval; and
a model component that estimates a remaining useful life of or recommends a maintenance task for at least one of the plurality of instrumented hardware parts, based on executing a deep learning neural network on the sequence of order-of-receipt knowledge graphs.

2. The system of claim 1, wherein the deep learning neural network comprises a graph encoder that receives as input the sequence of order-of-receipt knowledge graphs and computes a respective embedding for each node of each one of the sequence of order-of-receipt knowledge graphs.

3. The system of claim 2, wherein the deep learning neural network comprises a graph convolutional layer downstream of the graph encoder, and wherein, for each order-of-receipt knowledge graph in the sequence of order-of-receipt knowledge graphs, the graph convolutional layer performs a convolution between: node embeddings produced by the graph encoder for that order-of-receipt knowledge graph; and a kernel indicating to which of the plurality of instrumented hardware parts respective error codes belong.

4. The system of claim 3, wherein the deep learning neural network comprises an aggregation layer that is downstream of the graph convolutional layer, and wherein, for each order-of-receipt knowledge graph in the sequence of order-of-receipt knowledge graphs, the aggregation layer aggregates together convolved node embeddings produced by the graph convolutional layer for that order-of-receipt knowledge graph, thereby yielding a sequence of aggregated embeddings respectively corresponding to the sequence of order-of-receipt knowledge graphs.

5. The system of claim 4, wherein the aggregation layer aggregates convolved node embeddings via at least one from the group consisting of: a sum computation; an average computation; and a standard deviation computation.

6. The system of claim 4, wherein the deep learning neural network comprises a model head that receives the sequence of aggregated embeddings and computes as output an indicator indicating the remaining useful life or maintenance task.

7. The system of claim 1, wherein the medical imaging scanner comprises at least one from the group consisting of: a computed tomography scanner; an X-ray scanner; an ultrasound scanner; a magnetic resonance imaging scanner; a positron emission tomography scanner; a nuclear medicine scanner; and an image-guided surgery machine.

8. The system of claim 1, wherein the computer-executable components comprise:
a visualization component that renders on an electronic display:
an indication of the remaining useful life or maintenance task; and
a cyclicity diagram that is formed based on illustrating at least one of the sequence of order-of-receipt knowledge graphs such that its nodes are visually depicted in a circular layout.

9. The system of claim 1, wherein the sequence of time intervals is formed by scrubbing in defined temporal increments a sliding window across a time span measured backward from a current time.

10. A computer-implemented method, comprising:
accessing, by a device operatively coupled to a processor, a medical imaging scanner, wherein:
the medical imaging scanner comprises a plurality of instrumented hardware parts;
each of the plurality of instrumented hardware parts is monitored by a respective set of physical sensors; and
each of the plurality of instrumented hardware parts is configured to produce one or more respective electronic error codes in response to one or more respective combinations of measurements from its physical sensors;
constructing, by the device, a sequence of order-of-receipt knowledge graphs based on monitoring the plurality of instrumented hardware parts over a sequence of time intervals, wherein:
a first order-of-receipt knowledge graph of the sequence of order-of-receipt knowledge graphs corresponds to a first time interval of the sequence of time intervals;
each node of the first order-of-receipt knowledge graph represents a respective electronic error code producible by the plurality of instrumented hardware parts; and
each edge of the first order-of-receipt knowledge graph represents an order in which two respective electronic error codes are produced by the plurality of instrumented hardware parts during the first time interval; and
estimating or recommending, by the device, a remaining useful life of or a maintenance task for at least one of the plurality of instrumented hardware parts, based on executing a deep learning neural network on the sequence of order-of-receipt knowledge graphs.

11. The computer-implemented method of claim 10, wherein the deep learning neural network comprises a graph encoder that receives as input the sequence of order-of-receipt knowledge graphs and computes a respective embedding for each node of each one of the sequence of order-of-receipt knowledge graphs.

12. The computer-implemented method of claim 11, wherein the deep learning neural network comprises a graph convolutional layer downstream of the graph encoder, and wherein, for each order-of-receipt knowledge graph in the sequence of order-of-receipt knowledge graphs, the graph convolutional layer performs a convolution between: node embeddings produced by the graph encoder for that order-of-receipt knowledge graph; and a kernel indicating to which of the plurality of instrumented hardware parts respective error codes belong.

13. The computer-implemented method of claim 12, wherein the deep learning neural network comprises an aggregation layer that is downstream of the graph convolutional layer, and wherein, for each order-of-receipt knowledge graph in the sequence of order-of-receipt knowledge graphs, the aggregation layer aggregates together convolved node embeddings produced by the graph convolutional layer for that order-of-receipt knowledge graph, thereby yielding a sequence of aggregated embeddings respectively corresponding to the sequence of order-of-receipt knowledge graphs.

14. The computer-implemented method of claim 13, wherein the aggregation layer aggregates convolved node embeddings via at least one from the group consisting of: a sum computation; an average computation; and a standard deviation computation.

15. The computer-implemented method of claim 13, wherein the deep learning neural network comprises a model head that receives the sequence of aggregated embeddings and computes as output an indicator indicating the remaining useful life or maintenance task.

16. The computer-implemented method of claim 10, wherein the medical imaging scanner comprises at least one from the group consisting of: a computed tomography scanner; an X-ray scanner; an ultrasound scanner; a magnetic resonance imaging scanner; a positron emission tomography scanner; a nuclear medicine scanner; and an image-guided surgery machine.

17. The computer-implemented method of claim 10, further comprising:
rendering, by the device and on an electronic display:
an indication of the remaining useful life or maintenance task; and
a cyclicity diagram that is formed based on illustrating at least one of the sequence of order-of-receipt knowledge graphs such that its nodes are visually depicted in a circular layout.

18. The computer-implemented method of claim 10, wherein the sequence of time intervals is formed by scrubbing in defined temporal increments a sliding window across a time span measured backward from a current time.

19. A computer program product for facilitating failure prediction via graph representation of error codes, the computer program product comprising a non-transitory computer-readable memory having program instructions embodied therewith, the program instructions executable by a processor to cause the processor to:
access a machine, wherein:
the machine comprises a plurality of instrumented hardware parts;
each of the plurality of instrumented hardware parts is monitored by a respective set of physical sensors; and
each of the plurality of instrumented hardware parts is configured to produce one or more respective electronic error codes in response to one or more respective combinations of measurements from its physical sensors;
construct a sequence of knowledge graphs based on monitoring the plurality of instrumented hardware parts over a sequence of time intervals, wherein:
a first knowledge graph of the sequence of knowledge graphs corresponds to a first time interval of the sequence of time intervals;
each node of the first knowledge graph represents a respective electronic error code producible by the plurality of instrumented hardware parts; and
each edge of the first knowledge graph represents an order in which two respective electronic error codes are produced by the plurality of instrumented hardware parts during the first time interval; and
estimate or recommend a remaining useful life of or a maintenance task for at least one of the plurality of instrumented hardware parts, based on executing a deep learning neural network on the sequence of knowledge graphs.

20. The computer program product of claim 19, wherein the deep learning neural network convolves each of the sequence of knowledge graphs with a kernel indicating to which of the plurality of instrumented hardware parts respective error codes belong.
